(19) United States
(12) Patent Application Publication
(10) Pub. No.: US 2026/0337044 A1
(43) Pub. Date: Oct. 29, 2026

(54) Process for Production of Aluminium Hydroxide Particles

(71) Applicant: GlaxoSmithKline Biologicals SA, Rixensart (BE)

(72) Inventors: Francesco DORO, Siena (IT); Donatello LAERA, Siena (IT); Malte MEPPEN, Siena (IT); Bart RIMEZ, Lou-vain-la-Neuve (BE); Laurent STRODIOT, Rixensart (BE)

(73) Assignee: GlaxoSmithKline Biologicals SA, Rixensart (BE)

(21) Appl. No.: 19/115,312

(22) PCT Filed: Oct. 11, 2023
(86) PCT No.: PCT/EP2023/078254

§ 371 Date: Mar. 26, 2025

(30) Foreign Application Priority Data
Oct. 13, 2022 (GB) 2215072.6

(51) Int. Cl. C01F 7/34; A61K 39/00; A61K 39/39

(52) U.S. Cl. CPC C01F 7/34; A61K 39/39; A61K 2039/55505; C01P 2004/51

(57) ABSTRACT

The present invention relates inter alia to a method of making a composition comprising aluminium hydroxide particles wherein the method comprises reacting an aluminium ion-containing compound with a hydroxide ion-containing compound in a micro-fluidic or milli-fluidic (MF) system.

### TECHNICAL FIELD

The present invention relates to methods of making compositions comprising aluminium hydroxide particles and related concepts.

### BACKGROUND

Many vaccines require an adjuvant to induce a strong immune response. One such adjuvant is aluminium hydroxide. In an aqueous solution, aluminium hydroxide forms particles of around 1 to 20 um diameter as a result of aggregation. Aluminium hydroxide particles are traditionally produced by reacting aluminium chloride with sodium hydroxide in “batch mode”. Batch mode involves a step-by-step procedure for manufacturing the particles. Once one batch of particles is completed, the next batch begins, and so on. The production of each batch takes time and incurs costs. Accordingly, there is a need for alternative methods for the production of aluminium hydroxide particles, particularly methods which improve the time and cost efficiency of aluminium hydroxide particle production.

Furthermore, there is a need for methods which produce small aluminium hydroxide particles. It is clear that the size of particulate vaccine carriers significantly affects their adjuvant activities, and there are data showing that particulate vaccine carriers of around 200 nm (or less) may be optimal. Fifis et al. 2004 reported that ovalbumin (OVA)-conjugated polystyrene particles of 230 nm induced stronger OVA-specific antibody and cellular immune responses than other larger OVA-conjugated polystyrene particles after being intradermally injected into mice. Li et al. 2011 reported that small solid lipid nanoparticles of 200 nm have a more potent adjuvant activity than larger solid lipid nanoparticles of 700 nm, when OVA as an antigen is surface-conjugated on them. Li et al. 2013 reported the synthesis of aluminium hydroxide ‘nanoparticles’ with a mean diameter of 112 nm and compared their adjuvant activity with that of the traditional aluminium hydroxide suspension with a mean diameter of 9.3 um. It was found that protein antigens adsorbed on aluminium hydroxide nanoparticles induced a stronger antigen-specific antibody response than the same protein antigens adsorbed on the traditional aluminium hydroxide microparticles of around 9.3 um. It was also found that local inflammation induced by aluminium hydroxide nanoparticles in the injection sites was milder than that induced by microparticles. Particles of a size less than 1 um may be referred to as ‘nanoparticles’, or ‘nanoalum’.

The production of aluminium hydroxide nanoparticles therefore represents a desirable objective.

Additionally, from a regulatory perspective, clinical aluminium-based microparticles are not capable of being terminally sterilised by filtration through micron-sized filters (e.g. 0.8 um, 0.45 um or 0.22 um), and are only sterilisable by radiation or autoclave; making their manufacture not amenable to a terminal sterilisation step when combined with antigens or other adjuvants.

The characteristics of an ideal aluminium hydroxide particle-containing composition for use as a vaccine adjuvant are as follows. Firstly, a low particle size is desirable: ideally the particles have a mean diameter lower than 180 nm, such as wherein 99% or greater of the particle diameter distribution curve is below 180 nm. Secondly, the concentration of Al³⁺ in the composition is not less than about 0.3 mg/ml (i.e. not less than about 1 mg/ml Al(OH)₃). Thirdly, the pH of the composition is ideally between around 6.5 to 7.

### SUMMARY OF THE INVENTION

The present inventors have found that aluminium hydroxide particles can be produced by reacting an aluminium ion-containing compound with a hydroxide ion-containing compound in a micro-fluidic or milli-fluidic (MF) system. Suitably the aluminium ion-containing compound is AlCl₃ and the hydroxide ion-containing compound is NaOH. In some embodiments the production is substantially without interruption. In some embodiments, such continuous flow processes are more efficient and/or reduce waste compared to batch mode processes. The production volume may be adapted flexibly to meet demand (rather than a fixed volume for production as in batch mode). Furthermore, adaptation of the process from laboratory to manufacturing scale may be simplified: either elements of the system can be duplicated in parallel or the total flow rate may be increased.

Furthermore, the present inventors have found that the particles produced in this way may in at least some embodiments benefit from one or more of the favourable characteristics discussed above. As a result, the particles may find particular utility as adjuvants in vaccines.

In one aspect there is provided a method of making a composition comprising aluminium hydroxide particles wherein the method comprises reacting an aluminium ion-containing compound with a hydroxide ion-containing compound in a micro-fluidic or milli-fluidic (MF) system. In a further aspect there is provided a composition obtainable, such as obtained, by the method of the invention. In a further aspect there is provided an immunogenic composition comprising aluminium hydroxide particles obtained by the method of the invention. Also provided is the use of an MF system to prepare a composition comprising aluminium hydroxide particles.

Advantages of the embodiments of the invention relative to the prior art may be one or more of the following:
I. Faster production
II. Lower cost production
III. Continuous production
IV. Reduced waste
V. Reduced particle size
VI. Increased particle concentration
VII. Increased particle size consistency
VIII. Increased long term storage stability
IX. Increased adaptation of production volume to market needs
X. Increased production capacity
XI. Footprint reduction
XII. Reduced quantity of workers required
XIII. Reduction or elimination of liquid-air interface
XIV. Increased compatibility with process digitalisation
XV. Increased suitability for use as a vaccine adjuvant

### BRIEF DESCRIPTION OF THE FIGURES

FIG. 1 Particle size distribution of sample ALU.BRI.011 29

FIG. 2 Particle size distribution of sample ALU.BRI.011 30

FIG. 3 Particle size distribution of sample ALU.BRI.011 31

FIG. 4 Particle size distribution of sample ALU.BRI.011 32

FIG. 5 Particle size distribution of sample ALU-BRI-016 27 and 28

FIG. 6 Long term stability of samples ALU-BRI-019 15 and ALU-BRI-019 16

FIG. 7 MF system T cross schematic with example dimensions, mixing ratio and concentrations

FIG. 8 Effect of nanoalum or traditional aluminium hydroxide particles formulated with HlaCP5 antigen on total anti-Hla IgG induced following one or two immunisations (Example 9).

FIG. 9 Effect of nanoalum or traditional aluminium hydroxide particles formulated with HlaCP5 antigen on total anti-CP5 IgG induced following one or two immunisations (Example 9).

FIG. 10 Effect of nanoalum or traditional aluminium hydroxide particles formulated with HlaCP5 antigen on Hla antigen-specific CD4+ T cells induced following two immunisations (Example 9).

FIG. 11 Effect of nanoalum or traditional aluminium hydroxide particles formulated with HlaCP5 antigen on Hla antigen-specific CD8+ T cells induced following two immunisations (Example 9).

FIG. 12 Particle size distribution (relative to particle concentration) as determined by NTA (Example 10).

FIG. 13 Cryo-EM image at ×28000 magnification. Unfilled arrows indicate clustering of particles, filled arrows indicate smallest particles observed and the dashed arrow indicates the edge of the hole/carbon support.

FIG. 14 Cryo-EM image at 28000 magnification. 100 of the smallest particles observed were manually selected and a size estimation with a standard deviation was reported.

### DETAILED DESCRIPTION OF THE INVENTION

### Aluminium Hydroxide

### Aluminium Hydroxide Particles

‘Aluminium hydroxide’ as used herein refers to Al(OH)₃. Aluminium hydroxide particles in aqueous solution are formed from aggregates of individual aluminium hydroxide particles (‘primary’ particles). During storage, particularly when such aqueous solutions are static, the aluminium hydroxide particles can increase in size (i.e. aggregates bind together and/or bind with further primary particles). Suitably the particles comprise Al(OH)₃. More suitably the particles consist essentially of, or more suitably consist of, Al(OH)₃.

In one aspect the invention provides a method of making a composition comprising aluminium hydroxide particles wherein the method comprises reacting an aluminium ion-containing compound with a hydroxide ion-containing compound in a micro-fluidic or milli-fluidic (MF) system. Suitably, the aluminium ion-containing compound is comprised in an aqueous solution. More suitably, the solution essentially consists of an aluminium ion-containing compound and water, such as consists of an aluminium ion-containing compound and water. Suitably the hydroxide ion-containing compound is comprised in an aqueous solution. More suitably the solution essentially consists of a hydroxide ion-containing compound and water, such as consists of a hydroxide ion-containing compound and water.

Suitably an aluminium ion-containing compound comprises water of crystallisation, e.g. AlCl₃·6H₂O. Suitably AlCl₃·6H₂O used to prepare the aluminium hydroxide particles is comprised in an aqueous solution.

The molar ratio (also referred to herein as ‘molar excess’, ‘stoichiometric ratio’ or ‘stoichiometric excess’) of hydroxide ion-containing compound to an aluminium ion-containing compound in the reaction influences various factors including the size of the aluminium hydroxide particles, the concentration of aluminium hydroxide particles and the pH of the final composition. The hydroxide ion-containing compound over the aluminium ion-containing compound molar ratio is the number of moles of a hydroxide ion-containing compound divided by the number of moles of an aluminium ion-containing compound. In this context ‘over’ may also be expressed using the slash (/) symbol.

Suitably the molar ratio of the hydroxide ion-containing compound over the aluminium ion-containing compound is greater than 0.100, such as greater than 0.200, such as greater than 0.300, such as greater than 0.400, such as greater than 0.500, such as greater than 0.600, such as greater than 0.700, such as greater than 0.750, such as greater than 0.800, such as greater than 0.850, such as greater than 0.865.

Suitably the molar ratio of hydroxide ion-containing compound over aluminium ion-containing compound is no greater than 3.000, such as no greater than 2.500, such as no greater than 2.000, such as no greater than 1.500, such as no greater than 1.250, such as no greater than 1.000, such as no greater than 0.900, such as no greater than 0.880, such as no greater than 0.865.

Suitably the molar ratio of hydroxide ion-containing compound over aluminium ion-containing compound is 0.100 to 3.000, such as 0.200 to 2.500, such as 0.300 to 2.000, such as 0.500 to 1.500, such as 0.700 to 1.000, such as 0.800 to 0.900, such as about 0.865.

To produce desired molar ratios, different concentrations of aluminium ion-containing compound and hydroxide ion-containing compound may be used. Suitably the concentration of aluminium ion-containing compound is greater than 0.1 mg/ml, such as greater than 2 mg/ml, such as greater than 5 mg/ml, such as greater than 20 mg/ml, such as greater than 30 mg/ml, such as greater than 35 mg/ml, such as greater than 40.0 mg/ml.

Suitably the concentration of aluminium ion-containing compound is no greater than 1000 mg/ml, such as no greater than 500 mg/ml, such as no greater than 100 mg/ml, such as no greater than 75 mg/ml, such as no greater than 50 mg/ml, such as no greater than 45.0, such as no greater than 40.0 mg/ml.

Suitably the concentration of aluminium ion-containing compound is 0.1 mg/ml to 1000 mg/ml, such as 2 to 500 mg/ml, such as 5 mg/ml to 100 mg/ml, such as 30 mg/ml to 50 mg/ml, such as 35 to 45 mg/ml, such as about 40.0 mg/ml.

Suitably the concentration of hydroxide ion-containing compound is greater than 0.1 mg/ml, such as greater than 0.5 mg/ml, such as greater than 1 mg/ml, such as greater than 5 mg/ml, such as greater than 10 mg/ml, such as greater than 15 mg/ml, such as greater than 17.2 mg/ml.

Suitably the concentration of hydroxide ion-containing compound is no greater than 1000 mg/ml, such as no greater than 500 mg/ml, such as no greater than 80 mg/ml, such as no greater than 50 mg/ml, such as no greater than 25 mg/ml, such as no greater than 20 mg/ml, such as no greater than 17.2 mg/ml.

Suitably the concentration of hydroxide ion-containing compound is 0.1 mg/ml to 1000 mg/ml, such as 0.5 mg/ml to 500 mg/ml, such as 1 mg/ml to 80 mg/ml, such as 5 mg/ml to 50 mg/ml, such as 10 mg/ml to 25 mg/ml, such as 15 mg/ml to 20 mg/ml, such as about 17.2 mg/ml.

The mixing ratio of aluminium ion-containing compound over hydroxide ion-containing compound is the volume of fluid comprising the aluminium ion-containing compound entering the MF system per unit time divided by the volume of fluid comprising the hydroxide ion-containing compound entering the MF system per unit time. Suitably the mixing ratio of the aluminium ion-containing compound over the hydroxide ion-containing compound is 10/1 to 1/10, such as 5/1 to 1/5, such as 2/1 to 1/2, such as 1.5/1 to 1/1.5, such as about 1/1.

The volume of fluid entering the MF system is also referred to as the ‘flow rate’. Suitably the flow rate of the aluminium ion-containing compound (i.e. the flow rate of the fluid comprising the aluminium ion-containing compound) is 1 ml/min to 100 ml/min, such as 10 ml/min to 30 ml/min, such as 15 ml/min to 25 ml/min, such as about 20 ml/min. Suitably the flow rate of the hydroxide ion-containing compound (i.e. the flow rate of the fluid comprising the hydroxide ion-containing compound) is 1 ml/min to 100 ml/min, such as 10 ml/min to 30 ml/min, such as 15 ml/min to 25 ml/min, such as about 20 ml/min.

In certain embodiments, the concentration of the aluminium hydroxide produced by the methods of the invention is at least 2 mg/ml. In some embodiments, the concentration of the aluminium hydroxide is at least 5 mg/ml. In some embodiments, the concentration of the aluminium hydroxide is at least 9 mg/ml. In some embodiments, the concentration of the aluminium hydroxide is at least 12 mg/ml. In some embodiments, the concentration of the aluminium hydroxide is 0.5 to 50 mg/ml, 1 to 40 mg/ml, 2 to 30 mg/ml; 3 to 20 mg/ml or 4 to 15 mg/ml.

In certain embodiments, the size of the aluminium hydroxide particles is from about 50 nm to 75 nm. In some embodiments the size of the aluminium hydroxide particles is from about 50 nm to 100 nm. In some embodiments the size of the aluminium hydroxide particles is from about 50 nm to 150 nm. In some embodiments the size of the aluminium hydroxide particles is from about 50 nm to 200 nm. In some embodiments the size of the aluminium hydroxide particles is from about 50 nm to 300 nm. In some embodiments the size of the aluminium hydroxide particles is from about 50 nm to 400 nm. In some embodiments the size of the aluminium hydroxide particles is from about 50 nm to 450 nm. In certain embodiments, the size of the aluminium hydroxide particles is from about 70 nm to 75 nm. In some embodiments the size of the aluminium hydroxide particles is from about 70 nm to 100 nm. In some embodiments the size of the aluminium hydroxide particles is from about 70 nm to 150 nm. In some embodiments the size of the aluminium hydroxide particles is from about 70 nm to 200 nm. In some embodiments the size of the aluminium hydroxide particles is from about 70 nm to 300 nm. In some embodiments the size of the aluminium hydroxide particles is from about 70 nm to 400 nm. In some embodiments the size of the aluminium hydroxide particles is from about 70 nm to 450 nm.

In certain embodiments, the size of the aluminium hydroxide particles is about 1 nm, is about 5 nm, is about 10 nm, is about 15 nm, is about 20 nm, is about 25 nm, is about 30 nm, is about 35 nm, is about 40 nm, is about 45 nm, is about 50 nm, is about 55 nm, is about 60 nm, is about 65 nm, is about 70 nm, is about 75 nm, is about 80 nm, is about 85 nm, is about 90 nm, is about 95 nm, is about 100 nm, is about 105 nm, is about 110 nm, is about 115 nm, is about 120 nm, is about 125 nm, is about 130 nm, is about 135 nm, is about 140 nm, is about 145 nm, is about 150 nm, is about 155 nm, is about 160 nm, is about 165 nm, is about 170 nm, is about 175 nm, is about 180 nm, is about 185 nm, is about 190 nm, is about 195 nm, is about 200 nm, is about 210 nm, is about 220 nm, is about 240 nm, is about 250 nm, is about 260 nm, is about 280 nm, is about 290 nm, is about 300 nm, is about 320 nm, is about 340 nm, is about 350 nm, is about 360 nm, is about 380 nm, is about 400 nm, is about 420 nm, is about 440 nm, or is about 450 nm.

Most suitably the size of the aluminium hydroxide particles is no greater than about 180 nm, particularly no greater than 180 nm.

An average particle size can be measured. Accordingly, in certain embodiments, the average size of the aluminium hydroxide particles is about 1 nm-450 nm. In some embodiments the average size of the aluminium hydroxide particles ranges from about 50 nm to 75 nm. In some embodiments the average size of the aluminium hydroxide particles ranges from about 50 nm to 100 nm. In some embodiments the average size of the aluminium hydroxide particles ranges from about 50 nm to 150 nm. In some embodiments the average size of the aluminium hydroxide particles ranges from about 50 nm to 200 nm. In some embodiments the average size of the aluminium hydroxide particles ranges from about 50 nm to 300 nm. In some embodiments the average size of the aluminium hydroxide particles ranges from about 50 nm to 400 nm. In some embodiments the average size of the aluminium hydroxide particles ranges from about 50 nm to 450 nm. In some embodiments the average size of the aluminium hydroxide particles ranges from about 20 nm to 100 nm. In some embodiments the average size of the aluminium hydroxide particles ranges from about 20 nm to 50 nm. In some embodiments the average size of the aluminium hydroxide particles ranges from about 10 nm to 200 nm. In some embodiments the average size of the aluminium hydroxide particles ranges from about 10 nm to 100 nm. In some embodiments the average size of the aluminium hydroxide particles ranges from about 10 nm to 50 nm. In some embodiments the average size of the aluminium hydroxide particles is about 1 nm, is about 5 nm, is about 10 nm, is about 15 nm, is about 20 nm, is about 25 nm, is about 30 nm, is about 35 nm, is about 40 nm, is about 45 nm, is about 50 nm, is about 55 nm, is about 60 nm, is about 65 nm, is about 70 nm, is about 75 nm, is about 80 nm, is about 85 nm, is about 90 nm, is about 95 nm, is about 100 nm, is about 105 nm, is about 110 nm, is about 115 nm, is about 120 nm, is about 125 nm, is about 130 nm, is about 135 nm, is about 140 nm, is about 145 nm, is about 150 nm, is about 155 nm, is about 160 nm, is about 165 nm, is about 170 nm, is about 175 nm, is about 180 nm, is about 185 nm, is about 190 nm, is about 195 nm, is about 200 nm, is about 210 nm, is about 220 nm, is about 240 nm, is about 250 nm, is about 260 nm, is about 280 nm, is about 200 nm, is about 300 nm, is about 320 nm, is about 340 nm, is about 350 nm, is about 360 nm, is about 380 nm, is about 400 nm, is about 420 nm, is about 440 nm, or is about 450 nm.

In certain embodiments, the average size of the aluminium hydroxide particles is about 1 nm, no greater than about 5 nm, no greater than about 10 nm, no greater than about 15 nm, no greater than about 20 nm, no greater than about 25 nm, no greater than about 30 nm, no greater than about 35 nm, no greater than about 40 nm, no greater than about 45 nm, no greater than about 50 nm, no greater than about 55 nm, no greater than about 60 nm, no greater than about 65 nm, no greater than about 70 nm, no greater than about 75 nm, no greater than about 80 nm, no greater than about 85 nm, no greater than about 90 nm, no greater than about 95 nm, no greater than about 100 nm, no greater than about 105 nm, no greater than about 110 nm, no greater than about 115 nm, no greater than about 120 nm, no greater than about 125 nm, no greater than about 130 nm, no greater than about 135 nm, no greater than about 140 nm, no greater than about 145 nm, no greater than about 150 nm, no greater than about 155 nm, no greater than about 160 nm, no greater than about 165 nm, no greater than about 170 nm, no greater than about 175 nm, no greater than about 180 nm, no greater than about 185 nm, no greater than about 190 nm, no greater than about 195 nm, no greater than about 199 nm, no greater than about 200 nm, no greater than about 210 nm, no greater than about 230 nm, no greater than about 250 nm, no greater than about 270 nm, no greater than about 290 nm, no greater than about 310 nm, no greater than about 330 nm, no greater than about 350 nm, no greater than about 370 nm, no greater than about 390 nm, no greater than about 410 nm, no greater than about 430 nm, no greater than about 440 nm, or no greater than about 450 nm.

The mean diameter of particles in a solution may be characterised by a volume distribution. In some embodiments, the volume distribution mean diameter of the particles is 2.000 um or lower, such as 1.000 um or lower, such as 0.900 um or lower, such as 0.800 um or lower, such as 0.700 um or lower, such as 0.600 um or lower, such as 0.500 um or lower, such as 0.450 um or lower, such as 0.300 um or lower, such as 0.250 um or lower, such as 0.200 um or lower, such as 0.150 um or lower, such as 0.100 um or lower, such as 0.050 um or lower.

The mean diameter of particles in a solution may be characterised by a number distribution. In some embodiments, the number distribution mean diameter of the particles is 2.000 um or lower, such as 1.000 um or lower, such as 0.900 um or lower, such as 0.800 um or lower, such as 0.700 um or lower, such as 0.600 um or lower, such as 0.500 um or lower, such as 0.450 um or lower, such as 0.300 um or lower, such as 0.250 um or lower, such as 0.200 um or lower, such as 0.150 um or lower, such as 0.100 um or lower, such as 0.050 um or lower.

Suitably the number distribution mean diameter of the particles is 0.100 um or lower and the volume distribution mean diameter of the particles is 0.200 um or lower.

In some embodiments, the aluminium hydroxide particles are capable of being filtered through at least a 0.45 micron filter. In some embodiments, the aluminium hydroxide particles are capable of being filtered through a 0.45 micron or smaller pore size filter. In some embodiments, the aluminium hydroxide particles are capable of being filtered through a 0.45 micron filter. In some embodiments, the aluminium hydroxide particles are capable of being filtered through at least a 0.22 micron filter. In some embodiments, the aluminium hydroxide particles are capable of being filtered through a 0.22 micron or smaller pore size filter. In some embodiments, the aluminium hydroxide particles are capable of being filtered through a 0.22 micron filter. In some embodiments, the aluminium hydroxide particles are capable of being filtered through at least a 0.08 micron filter. In some embodiments, the aluminium hydroxide particles are capable of being filtered through a 0.08 micron or smaller pore size filter. In some embodiments, the aluminium hydroxide particles are capable of being filtered through a 0.08 micron filter.

Suitably the aluminium hydroxide particles are capable of being filtered through a 0.45 micron filter, followed by a 0.22 micron filter, followed by a 0.08 micron filter.

Uniformity of particle sizes is desirable. A polydispersity index (Pdl) of greater than 0.7 indicates that the sample has a very broad size distribution and a reported value of 0 means that size variation is absent, although values smaller than 0.05 are rarely seen. Suitably the aluminium hydroxide particles have a polydispersity of 0.5 or less, especially 0.3 or less, such as 0.2 or less.

Suitably 70% or greater, such as 75% or greater, such as 80% or greater, such as 85% or greater, such as 90% or greater, such as 95% or greater of the particle diameter distribution curve is below a particle diameter recited above. Most suitably 99% or greater of the particle diameter distribution curve is below 180 nm diameter.

The particle size, as used herein, means the average diameter of particles (in an aqueous solution) and can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which size is determined. The preferred method for defining the average particle diameter is a Z-average i.e. the intensity-weighted mean hydrodynamic size of the ensemble collection of particles measured by DLS. The Z-average is derived from cumulants analysis of the measured correlation curve, wherein a single particle size (diameter) is assumed and a single exponential fit is applied to the autocorrelation function. Thus, references herein to average particle size should be taken as an intensity-weighted average, and ideally the Z-average. Pdl values are easily provided by the same instrumentation which measures average diameter. Unless otherwise stated, the sizes described herein refer to the Z-average.

The particle size may also be measured using cryogenic electron microscopy (cryo-EM). In certain embodiments, the average size of the aluminium hydroxide particles is about 1 nm, no greater than about 5 nm, no greater than about 10 nm, no greater than about 15 nm, no greater than about 20 nm, no greater than about 25 nm, no greater than about 30 nm, no greater than about 35 nm, no greater than about 40 nm, no greater than about 45 nm, no greater than about 50 nm, no greater than about 55 nm, no greater than about 60 nm, no greater than about 65 nm, no greater than about 70 nm, no greater than about 75 nm, no greater than about 80 nm, no greater than about 85 nm, no greater than about 90 nm, no greater than about 95 nm, no greater than about 100 nm, no greater than about 105 nm, no greater than about 110 nm, no greater than about 115 nm, no greater than about 120 nm, no greater than about 125 nm, no greater than about 130 nm, no greater than about 135 nm, no greater than about 140 nm, no greater than about 145 nm, no greater than about 150 nm, no greater than about 155 nm, no greater than about 160 nm, no greater than about 165 nm, no greater than about 170 nm, no greater than about 175 nm, no greater than about 180 nm, no greater than about 185 nm, no greater than about 190 nm, no greater than about 195 nm, no greater than about 199 nm, no greater than about 200 nm, no greater than about 210 nm, no greater than about 230 nm, no greater than about 250 nm, no greater than about 270 nm, no greater than about 290 nm, no greater than about 310 nm, no greater than about 330 nm, no greater than about 350 nm, no greater than about 370 nm, no greater than about 390 nm, no greater than about 410 nm, no greater than about 430 nm, no greater than about 440 nm, or no greater than about 450 nm as measured by cryo-EM.

The zeta potential (ZP) of the aluminium hydroxide particles may be measured. Suitably the aluminium hydroxide particles have a ZP of lower than 0 mV, such as lower than-10 mV, such as lower than-20 mV, such as lower than-30 mV, such as about-40 mV.

### Aluminium Ion-Containing Compound

The aluminium ion-containing compound is capable of reacting with the hydroxide ion-containing compound to produce aluminium hydroxide in aqueous solution. The anionic group in the aluminium ion-containing compound is such that the compound forms aluminium ions in aqueous solution.

Ideally the aluminium ion-containing compound is pharmaceutically acceptable (e.g. it should be suitable for injection to a subject without significant detrimental effects).

In one embodiment, the anionic group in the aluminium ion-containing compound is selected from chloride or sulfate. In one embodiment, the aluminium ion-containing compound is selected from the group consisting of KAl(SO₄)₂, AlCl₃, NH₄Al(SO₄)₂ and Al₂(SO₄)₃. Most suitably the aluminium ion-containing compound is aluminium chloride (AlCl₃).

### Hydroxide Ion-Containing Compound

The hydroxide ion-containing compound is capable of reacting with the aluminium ion-containing compound to produce aluminium hydroxide in aqueous solution. The cationic group in the hydroxide ion-containing compound is such that the compound forms hydroxide ions in aqueous solution.

Ideally the hydroxide ion-containing compound is pharmaceutically acceptable (e.g. it should be suitable for injection to a subject without significant detrimental effects).

In one embodiment, the cationic group in the hydroxide ion-containing compound is selected from group 1 metal ions or ammonium ions. Suitably, the hydroxide ion-containing compound is selected from the group consisting of NaOH, KOH and NH₄OH. More suitably the hydroxide ion-containing compound is NaOH.

### Aqueous Compositions Comprising Aluminium Hydroxide Particles

Suitably the aluminium ion-containing compound and hydroxide ion-containing compound are comprised in aqueous solutions. Suitably one aqueous solution comprises, such as essentially consists of, such as consists of an aluminium ion-containing compound dissolved in water and one aqueous solution comprises, such as essentially consists of, such as consists of a hydroxide ion-containing compound dissolved in water. Suitably, no further components are substantially present in these aqueous solutions. The composition produced by the method of the invention is suitably a suspension of aluminium hydroxide particles in water. Suitably no further components are introduced into the MF system.

Suitably neither hydrochloric acid nor citric acid are added to the aluminium ion-containing compound solution or hydroxide ion-containing compound solution before the reaction. More suitably acid is not added to the aluminium ion-containing compound solution or hydroxide ion-containing compound solution before the reaction.

A pharmaceutically acceptable osmolality will generally mean that solutions will have an osmolality which is approximately isotonic or mildly hypertonic. Suitably the compositions of the present invention when reconstituted will have an osmolality in the range of 150 to 750 mOsm/kg, for example, the osmolality may be in the range of 200 to 400 mOsm/kg, such as in the range of 240 to 360 mOsm/kg. Osmolality may be measured according to techniques known in the art, such as by the use of a commercially available osmometer, for example the Advanced™ Model 2020 available from Advanced Instruments Inc. (USA).

Suitably the method of the invention comprises the additional step of adjusting the osmolarity of the composition to the ranges quoted above, such as 240 to 360 mOsm/kg.

A tonicity modifier may be introduced to the composition. The tonicity of the composition may be adjusted with a suitable tonicity modifier. Most suitably the tonicity is modified with sucrose. Suitably the concentration of the sucrose is 4 to 12% w/v, such as 6 to 10% w/v, such as about 8% w/V.

A buffer may be introduced to the composition. Most suitably the buffer is histidine. Suitably the concentration of the histidine buffer is 5 to 15 mM, such as 7 to 13 mM, such as about 10 mM. The histidine buffer suitably has a pH of 6 to 7, such as about 6.5.

Suitably the aluminium hydroxide particles will be present such that the concentration of Al³⁺ is at least 0.5 mg/ml (e.g. at least 1 μg/ml, at least 2 μg/ml, at least 3 μg/ml etc.).

Suitably the compositions of the invention have a pH of 6.0 to 7.5, such as 6.5 to 7.0. Suitably the methods of the invention comprise the additional step of adjusting the pH of the composition to 6.0 to 7.5, such as 6.5 to 7.0.

### Aluminium Hydroxide Particle Stability

In some embodiments provided herein, the size of the aluminium hydroxide particles is stable, in that the aluminium hydroxide particles' size is maintained, and in that the aluminium hydroxide particles exhibit reduced aggregation, or no aggregation, when compared to aluminium hydroxide particles produced by means of the prior art.

“Stable” refers to a composition comprising aluminium hydroxide particles which do not aggregate, displays little to no aggregation, or reduced aggregation and/or demonstrate little to no overall increase in average particle size or polydispersity of the formulation over time compared to the starting particle size (i.e. the size of the particles when first formed).

The stability of the aluminium hydroxide particles can be measured by techniques familiar to those of skill in the art. In some embodiments, the stability is observed visually. Visual inspection can include inspection for particulates, flocculence, cloudiness or aggregates. In some embodiments, the stability is determined by the size of the aluminium hydroxide particles, suitably measured according to the particle size measurement methods described above. For example, the size can be assessed by known techniques in the art, including but not limited to, x-ray and laser diffraction, dynamic light scattering (DLS) or CryoEM. In some embodiments, the size of the aluminium hydroxide particles refers to the Z-average diameter, suitably established using DLS such as using a Malvern Zetasizer.

In some embodiments, stability is assessed by the ability of the aluminium hydroxide particles to pass through a filter of a particular size, for example through a 0.8 um, or 0.22 um or 0.45 um filter. In some embodiments, stability is determined by measurement of the polydispersity index (Pdl), for example with the use of the dynamic light scattering (DLS) technique.

In some embodiments, the Z-average diameter of the nanoparticle increases less than 50%, less than 40%, less than 30%, less than 25%, less than 20%, less than 15%, less than 12%, less than 10%, less than 7%, less than 5%, less than 3%, less than 1% over the time period assayed.

In some embodiments, the polydispersity index (Pdl) of the nanoparticle increases less than 50%, less than 40%, less than 30%, less than 25%, less than 20%, less than 15%, less than 12%, less than 10%, less than 7%, less than 5%, less than 3%, less than 1% over the time period assayed. Suitable time periods assayed are, for example at least 5 minutes, for at least minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years.

In some embodiments, the aluminium hydroxide particles are stable at below 20 °C, such as below 15 °C, such as below 10, such as below 8 °C, such as 2 to 8 °C.

In some embodiments, the aluminium hydroxide particles are stable at below 20 °C, such as below 15 °C, such as below 10 °C, such as below 8 °C, such as 2 to 8 °C for at least 1 minute, for at least 5 minutes, for at least 10 minutes, for at least 15 minutes, for at least 20 minutes, for at least 25 minutes, for at least 30 minutes, for at least 35 minutes, for at least 40 minutes, for at least 45 minutes, for at least 50 minutes, for at least 55 minutes, for at least 1 hour, for at least 2 hours, for at least 6 hours, for at least 12 hours, for at least 18 hours, for at least 24 hours, for at least 48 hours, for at least 72 hours, for at least 1 week, for at least 2 weeks, for at least 3 weeks, for at least 1 month, for at least 2 months, for at least 3 months, for at least 4 months, for at least 5 months, for at least 6 months, for at least 7 months, for at least 8 months, for at least 9 months, for at least 10 months, for at least 11 months, for at least 1 year, for at least 2 years, or for at least 5 years.

Most suitably, the aluminium hydroxide particles are substantially stable (suitably at 2 to 8 °C) for at least 200 days, such as at least 180 days, such as at least 150 days, such as at least 100 days, such as at least 50 days, such as at least 30 days, such as at least 20 days, such as at least 10 days.

In some embodiments, the compositions are suitable for filtering prior to vialing. The filter is suitably of a pore size such that the compositions are sterilised by filtering. In some embodiments, the composition is capable of being filtered through a 0.8 micron filter. More suitably, the composition is capable of being filtered through a 0.45 micron filter. More suitably, the composition is capable of being filtered through a 0.22 micron filter.

### Micro-Fluidic or Milli-Fluidic (MF) Systems

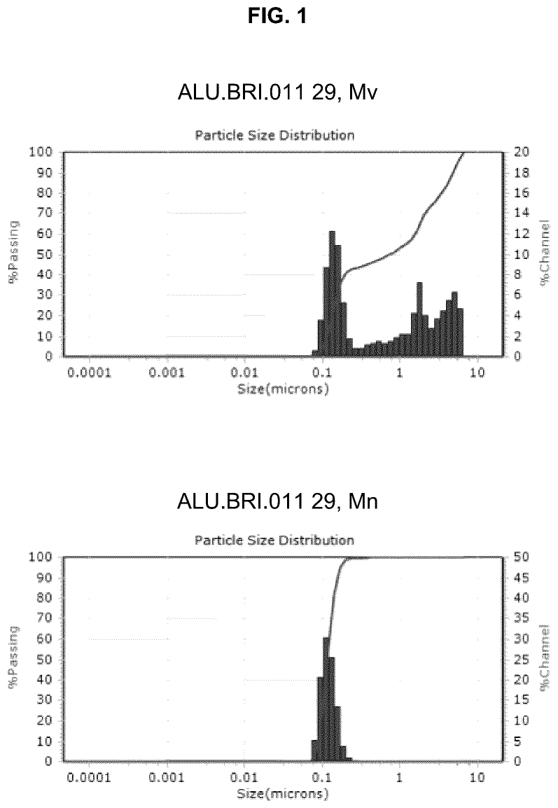

A micro-fluidic or milli-fluidic (MF) system is a fluid handing apparatus typically having dimensions on a um (micro-fluidic) or mm (milli-fluidic) scale and typically mixing occurs through passive means (i.e. through contact of fluid streams and without moving parts within the mixer). In some embodiments, pumps may propel fluids (e.g. aqueous solution comprising aluminium ion-containing compound and aqueous solution comprising hydroxide ion-containing compound) into and through the MF system. A suitable MF system for implementing the invention is that disclosed in WO2019215022 (specifically FIG. 1). WO2019215022 is incorporated by reference herein in its entirety for the purpose of the details of MF systems disclosed therein.

As used herein, in “fluid communication” means that the component(s) is/are structurally arranged to allow passage of fluid (e.g., a first inlet in fluid communication with a line means that fluid may flow via the line to the first inlet).

As used herein, a “line” may be a continuous supply of a fluid, such as water to the MF system. In some aspects, the line may include tubing for supplying the fluid.

As used herein, an “inlet” refers to the portion of the micromixer that receives fluid input. Inlets may merge prior to feeding fluids into a channel for mixing. As used herein, an “outlet” refers to the portion of the mixer that provides a processed fluid.

As used herein, a “fluid” may refer to any suitable liquid, buffer, solution, etc. used in the production of the composition comprising aluminium hydroxide particles.

As used herein, a “reservoir” may be any suitable container for holding a fluid to be provided to the mixer.

As used herein, a “channel” refers to a portion of the mixer where mixing occurs. The channel may have an input fluidly connected to the mixer inlets and an output fluidly connected to the micromixer outlet. It is understood that the mixer provides a path for fluid flow from the inlets to the outlet.

The MF system may be formed from any suitable material, in particular one which is tolerant of the one or more constituents introduced into the system, such as AlCl₃ or NaOH. Suitable materials include plastic, silicon, glass and stainless steel. Systems may be prepared from such materials by etching, e.g. silicon systems may be prepared by Deep Reactive Ion Etching (DRIE or plasma etching), glass systems may be prepared by wet etching (HF etching) and plastic systems may be prepared by 3D printing. Chosen materials may be subjected to surface treatment to improve the characteristics of the surface.

The method of the invention comprises reacting an aluminium ion-containing compound with a hydroxide ion-containing compound in a micro-fluidic or milli-fluidic (MF) system. In one embodiment, the MF system is a micro-fluidic system. In an alternative embodiment, the MF system is a milli-fluidic system. Suitably the flow path of the fluids throughout the MF system has a diameter of no greater than 900 micrometers, such as no greater than 700 micrometers, such as no greater than 500 micrometers.

### Mixers

A dedicated mixer is not required if the aluminium ion-containing compound and hydroxide ion-containing compound are substantially completely mixed and reacted before exiting the MF system. Such mixing and reaction may occur simply by virtue of turbulence in the MF system. The requirement for a dedicated mixer will depend on parameters such as the size and shape of the MF system.

The MF system may comprise one or more mixers within which the aluminium ion-containing compound and hydroxide ion-containing compound are mixed before subsequently reacting to produce Al(OH)₃ (and NaCl). Suitably the one or more mixers comprise a mixing channel having a shape which induces turbulence in the flow of the liquid, facilitating mixing and complete reaction.

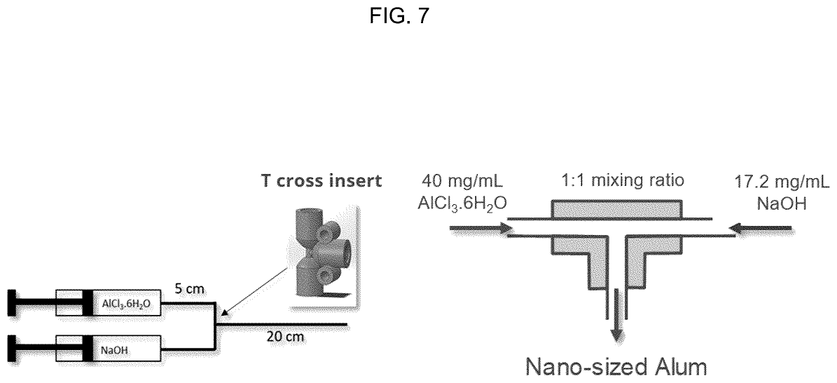

Suitably the one or more mixers are micromixers. Micromixers are mixers suitable for use in MF systems. Micromixers suitable for use in the present invention may be static (passive) or dynamic (active). Static mixing involves the mixing of components without the application of external forces, i.e. using solely the movement of the components through the MF system to achieve mixing. A pump may propel fluid into an input of the micromixer, but the mixing is still considered passive within the channel. Ideally such mixing will be enhanced by introducing a complex route through the MF system in a mixing chamber. Static mixing approaches include Y- and T-type flow-, multi-laminating-, split-and-recombine-, chaotic-, jet colliding- and recirculation flow-mixers. T-type flow is achieved using a T-mixer (or ‘T-cross’, see for a particular example FIG. 7), wherein the aluminium ion-containing compound and hydroxide ion-containing compound come into contact with each other by substantially opposite flow directions meeting, and the resultant mixture escaping by a substantially right-angled escape channel.

Dynamic mixing involves the application of external forces, such as stirrers. Dynamic mixing approaches include acoustic fluid shaking (such as using sonicators), ultrasound (such as using ultra-sonicators), electrowetting-based droplet shaking and microstirrers.

Most suitably the micromixers are static micromixers. Suitably the one or more mixers are not dynamic (active) mixers. In one embodiment, the mixers do not comprise moving parts. In one embodiment the mixers are not sonicators.

Desirably the flow rates measured in each mixing chamber vary by less than 5% from the desired flow rate.

In one embodiment the MF system may comprise a reactor. A reactor may be required if the reaction is not substantially completed in the mixer. The reactor is a portion of the MF system in which the combined aluminium ion-containing compound and hydroxide ion-containing compound travel and, while doing so, complete their reaction to produce Al(OH)₃ (and any by-product(s)). Suitably the aluminium ion-containing compound and hydroxide ion-containing compound are reacted in a reactor having a volume of 0.1 to 1 ml, such as 0.4 to 0.6 ml, such as about 0.5 ml. The reactor chamber comprised within the reactor should be of adequate length to allow for mixing to be substantially complete and a substantially homogenous mixture produced. Typically, the chamber will be 1-500 cm in length, such as 5-300 cm, especially 10-250 cm, in particular 15-200 cm, for example 20-100, such as about 50 cm. Typically, the diameter of the fluid flow path within the chamber will be approximately 500 micrometers.

### Inlets and Outlets

The MF system suitably comprises at least two inlets. One inlet for introduction of the aluminium ion-containing compound and one inlet for introduction of the hydroxide ion-containing compound. Suitably the MF system comprises five or fewer inlets, such as four or fewer inlets, for example three or fewer inlets, such as two inlets. Alternatively, the MF system will comprise three or more inlets, such as four or more inlets, for example five or more inlets.

The MF system may comprise at least one valve controlling removal of the composition from the MF system by filling one or more vessels with the composition or by diversion into a waste container. The system may have a plurality of valves/outlets for recovery of the composition, such as two or three outlets. Most suitably the system will have a single outlet.

It is desirable for the system to achieve a sufficient level of productivity. Additionally, to reduce the impact of startup and shutdown effects it is necessary for the run time to be of adequate length (e.g. at least 30 minutes, especially at least 60 minutes).

The aluminium ion-containing compound and hydroxide ion-containing compound may be stored in separate containers and introduced into the system directly from these separate containers.

### Operating Conditions

Optimal operating conditions will depend on the precise configuration of the device and the desired characteristics of the composition. Suitably, the flow rate of the aluminium ion-containing compound is 1 ml/min to 100 ml/min, such as 10 ml/min to 30 ml/min, such as 15 ml/min to 25 ml/min, such as about 20 ml/min. Suitably, the flow rate of the hydroxide ion-containing compound is 1 ml/min to 100 ml/min, such as 10 ml/min to 30 ml/min, such as 15 ml/min to 25 ml/min, such as about 20 ml/min.

The aluminium ion-containing compound and hydroxide ion-containing compound will typically be provided at a temperature in the region of 10-30 °C, such as 15-25 °C, in particular 18-22 °C especially 20 °C), and may be at the same or different temperatures, suitably at the same temperature and especially at 20 °C. The MF system may be maintained at a temperature in the region of 10-30 °C, such as 15-25 °C, in particular 18-22 °C, especially 20 °C. Dependent on the design of the system and environmental conditions it may only be necessary to actively control the temperature of the one or more constituents, and not to actively control the MF system temperature. The MF system may be operated within a controlled temperature environment, e.g. where the temperature is maintained in the range of 10-30 °C, such as 15-25 °C, in particular about 20 °C (such as 18-22 °C, in particular 20 °C).

The operating pressure of the system may be controlled. Suitably, the maximum pressure within the system may be 5 bar, such as 2 bar, such as 1 bar, such as 0.8 bar, such as 0.7 bar, such as 0.6 bar.

### Pumps

The aluminium ion-containing compound and hydroxide ion-containing compound may be introduced into the MF system by any suitable means. Most suitably the aluminium ion-containing compound and hydroxide ion-containing compound are introduced into the MF system by one or more pumps. Typically each constituent is introduced by a separate pump. Suitable pumps include syringe pumps, gear pumps and piston pumps. It is desirable to use pumps which are adequately precise and substantially pulsation-free. For convenience, it is also desirable for the pumps to be electronically-controlled, such as computer-controlled.

If pumps are used, typically each constituent will be introduced into the MF system by one pump per constituent. The optimal flow rate may depend on factors including the diameter of the tubing, the number of mixers and the concentration of the constituents. Suitably, the one or more pumps operate at a pressure of at least 5 bar, such as at least 2 bar, such as at least 1 bar, such as at least 0.8 bar, such as at least 0.7 bar, such as at least 0.6 bar.

The use of an MF system to produce aluminium hydroxide particles permits continuous production of the particles. In one embodiment, the composition is made in a continuous flow process.

To begin operation of the MF system, the operator typically will initially prime the system by commencing continuous introduction of the aluminium ion-containing compound and hydroxide ion-containing compound, firstly allowing air to escape from the system and latterly allowing waste such as improperly reacted aluminium ion-containing compound and/or hydroxide ion-containing compound to be removed (‘waste priming’, for example by a priming valve or by collection in a waste vessel), until the composition from the output consists of the desired concentration of Al(OH)₃. At this stage the continuous flow process has begun.

A “continuous” production process (or “continuous flow process”) is a process in which the product is produced without interruption. In the present context, the term is to be interpreted to refer to the substantially uninterrupted transfer of constituents through the steps of the process. The movement of the constituents in the system is such that the constituents do not substantially remain in one location in the system, or at one step, for a significant or undesired period of time. The process does not require upstream steps to run to completion before downstream steps may commence. When the continuous flow process is underway, constituents are being introduced into the system at the same time final product is being removed from the system.

To prevent interference with the reaction, it may be advantageous to introduce no further constituents into the MF system for the reaction, other than an aluminium ion-containing compound and a hydroxide ion-containing compound (both suitably in aqueous solution).

### Filters

Suitably the MF system comprises a filter (e.g. a sterilizing filtration unit). For parenteral administration in particular, compositions produced by the process of the invention should be sterile. Sterilisation can be performed by various methods although is conveniently undertaken by filtration through a sterile grade filter. By “sterile grade filter” it is meant a filter that produces a sterile effluent after being challenged by microorganisms at a challenge level of greater than or equal to 1×10⁷/cm² of effective filtration area. Sterile grade filters are well known to the person skilled in the art of the invention. For the purpose of the present invention, sterile grade filters have a pore size of 0.1-0.5 um, suitably 0.18-0.22 um, such as 0.2 or 0.22 um. The membranes of the sterile grade filter can be made from any suitable material known to the skilled person, for example, but not limited to cellulose acetate, polyethersulfone (PES), polyvinylidene fluoride (PVDF), polytetrafluoroethylene (PTFE).

### Sensors

Suitably one or more sensors are included at the outlet or one or more inlets of the MF system. The sensor(s) suitably monitor one or more of pH, temperature or pressure. Most suitably the sensors monitor pressure.

If pumps and sensors are utilised in the MF system, then suitably these components may be monitored and controlled automatically or partly automatically using a computer. Suitably the computer is a Process Analytical Technology (PAT) device.

### Subsequent Processing

One or more further processing steps may be carried out after the composition has been produced using the MF system.

### Dialysis

After the composition has been produced using the MF system, the composition may comprise one or more undesirable components. Such undesirable components may be removed, or the concentration of these components significantly reduced, by dialysis. Accordingly, in one embodiment the composition is dialysed.

In particular, if a stoichiometric excess of aluminium ion-containing compound is used in the reaction, then excess unreacted aluminium ion-containing compound will remain in the composition after reaction completion. In one embodiment, the unreacted aluminium ion-containing compound may be partially or completely removed. Suitably partial or complete removal is performed by dialysis. Accordingly, in one embodiment the composition is dialysed to reduce the concentration of any unreacted aluminium ion-containing compound, such as to remove substantially all unreacted aluminium ion-containing compound. Alternatively, or in addition, an undesirable concentration of NaCl may be present in the composition after production in the MF system (NaCl is a by-product of the reaction). Accordingly, in one embodiment the composition is dialysed to reduce the concentration of NaCl. Suitably dialysis uses a dialysis membrane having a molecular cutoff of 2.5 to 4.5 KD, such as about 3.5 KD.

Any suitable dialysis medium may be used. However, most suitably a citric acid buffer is used as the dialysis medium. Suitably the dialysis medium is added to the aluminium hydroxide composition and the surrounding solution (i.e. both inside and outside the dialysis membrane). Suitably the citric acid buffer has a concentration of 5 to 50 mM, such as 10 to 30 mM, such as about 20 mM. Alternatively the citric acid buffer has a concentration of 5 to 25 mM, such as 10 to 20 mM. Alternatively the citric acid buffer has a concentration of 15 to 50 mM, such as 20 to 50 mM. Suitably the citric acid buffer has a concentration of greater than 5 mM, such as greater than 15 mM. Suitably the citric acid buffer has a contraction of less than 50 mM, such as less than 25 mM. Suitably the citric acid buffer has a pH of 4.0 to 6.9, such as 5.3 to 6.7, such as 5.5 to 6.5.

The citric acid buffer comprises (such as consists of) citric acid and a compound providing a counter-ion which provides the desired buffering capacity. Suitable compounds providing counter-ions include those having a similar pKa to sodium citrate, such as a pKa of 1 to 4 or more suitably 2 to 3, such as about 2.5. Suitably the compound providing the counter-ion is selected from the group consisting of lithium citrate, sodium citrate, potassium citrate and rubidium citrate; and hydrates thereof. More suitably sodium citrate or potassium citrate and hydrates thereof, most suitably sodium citrate and hydrates thereof, specifically sodium citrate dihydrate.

Citric acid buffers comprising citric acid and sodium citrate dihydrate may be prepared as follows (“Citrate Buffer (pH 3.0 to 6.2) Preparation.” AAT Bioquest, Inc, 3 Jun. 2021, https://www.aatbio.com/resources/buffer-preparations-and-recipes/citrate-buffer-ph-3-to-6-2.)

| Required | Concentration/ | Citric | Sodium citrate |
|---|---|---|---|
| pH | mM | acid (g) | dihydrate (g) |
| 5.7 | 20 | 4.763 | 22.118 |
| 6.0 | 20 | 3.358 | 24.269 |
| 6.5 | 20 | 2.421 | 25.703 |

Note:
for a required pH of 6.5, pH needs to be adjusted with NaOH.

Suitably after dialysis the pH of the composition is 6.0 to 7.5, such as 6.5 to 7.0.

### Filtration

Suitably, after the composition has been produced in and removed from the MF system, the composition is filtered. If the composition has been dialysed, then suitably filtration is performed after dialysis. Filtration may be performed to sterilise the composition and/or to further reduce and/or make more consistent the size of the aluminium hydroxide particles. If dialysis is also performed, then suitably the composition is filtered after dialysis. Suitably the composition is filtered with a 0.45 um filter and/or a 0.22 um filter.

Suitably filtration reduces the size of the aluminium hydroxide particles. Suitably the volume distribution mean diameter of the particles after filtration is 1.000 um or lower, such as 0.800 um or lower, such as 0.500 um or lower, such as 400 um or lower, such as 0.300 um or lower, such as 0.250 um or lower, such as 0.200 um or lower, such as 0.100 um or lower.

Suitably the number distribution mean diameter of the particles after filtration is 1.000 um or lower, such as 0.800 um or lower, such as 0.500 um or lower, such as 400 um or lower, such as 0.300 um or lower, such as 0.250 um or lower, such as 0.200 um or lower, such as 0.100 um or lower.

### Miscellaneous

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

### CLAUSES

Clauses further illustrating the invention are as follows:
1. A method of making a composition comprising aluminium hydroxide particles wherein the method comprises reacting an aluminium ion-containing compound with a hydroxide ion-containing compound in a micro-fluidic or milli-fluidic (MF) system.
2. The method of clause 1 comprising reacting an aluminium ion-containing compound with a hydroxide ion-containing compound in a micro-fluidic system.
3. The method of clause 1 comprising reacting an aluminium ion-containing compound with a hydroxide ion-containing compound in a milli-fluidic system.
4. The method of any one of clauses 1 to 3 wherein the aluminium ion-containing compound is pharmaceutically acceptable.
5. The method of any one of clauses 1 to 4 wherein the aluminium ion-containing compound is selected from chloride or sulfate.
6. The method of clause 5 wherein the aluminium ion-containing compound is selected from the list consisting of KAl(SO₄)₂, AlCl₃, NH₄Al(SO₄)₂ and Al₂(SO₄)₃.
7. The method of clause 6 wherein the aluminium ion-containing compound is AlCl₃.
8. The method of any one of clauses 1 to 7 wherein the hydroxide ion-containing compound is pharmaceutically acceptable.
9. The method of any one of clauses 1 to 8 wherein the hydroxide ion-containing compound is selected from group 1 metal ions or ammonium ions.
10. The method of clause 9 wherein the hydroxide ion-containing compound is selected from the list consisting of NaOH, KOH and NH₄OH.
11. The method of clause 10 wherein the hydroxide ion-containing compound is NaOH.
12. The method of any one of clauses 1 to 11 wherein the aluminium ion-containing compound is comprised in an aqueous solution.
13. The method of clause 12 wherein the solution essentially consists of an aluminium ion-containing compound and water.
14. The method of clause 13 wherein the solution consists of an aluminium ion-containing compound and water.
15. The method of any one of clauses 1 to 14 wherein the hydroxide ion-containing compound is comprised in an aqueous solution.
16. The method of clause 15 wherein the solution essentially consists of a hydroxide ion-containing compound and water.
17. The method of clause 16 wherein the solution consists of a hydroxide ion-containing compound and water.
18. The method of any one of clauses 1 to 17 wherein the molar ratio of the hydroxide ion-containing compound/the aluminium ion-containing compound is greater than 0.100.
19. The method of clause 18 wherein the molar ratio of the hydroxide ion-containing compound over the aluminium ion-containing compound is greater than 0.300.
20. The method of clause 19 wherein the molar ratio of the hydroxide ion-containing compound over the aluminium ion-containing compound is greater than 0.700.
21. The method of clause 20 wherein the molar ratio of the hydroxide ion-containing compound over the aluminium ion-containing compound is greater than 0.800.
22. The method of clause 21 wherein the molar ratio of the hydroxide ion-containing compound over the aluminium ion-containing compound is greater than 0.865.
23. The method of any one of clauses 1 to 22 wherein the molar ratio of the hydroxide ion-containing compound over the aluminium ion-containing compound is no greater than 3.000.
24. The method of clause 23 wherein the molar ratio of the hydroxide ion-containing compound over the aluminium ion-containing compound is no greater than 2.000.
25. The method of clause 24 wherein the molar ratio of the hydroxide ion-containing compound over the aluminium ion-containing compound is no greater than 1.000.
26 The method of clause 25 wherein the molar ratio of the hydroxide ion-containing compound over the aluminium ion-containing compound is no greater than 0.900.
27 The method of clause 26 wherein the molar ratio of the hydroxide ion-containing compound over the aluminium ion-containing compound is no greater than 0.87.
28 The method of any one of clauses 1 to 17 wherein the molar ratio of the hydroxide ion-containing compound over the aluminium ion-containing compound is 0.100 to 3.000.
29. The method of clause 28 wherein the molar ratio of the hydroxide ion-containing compound over the aluminium ion-containing compound is 0.300 to 2.000.
30 The method of clause 29 wherein the molar ratio of the hydroxide ion-containing compound over the aluminium ion-containing compound is 0.700 to 1.000.
31 The method of clause 30 wherein the molar ratio of the hydroxide ion-containing compound over the aluminium ion-containing compound is 0.800 to 0.900.
32 The method of clause 31 wherein the molar ratio of the hydroxide ion-containing compound over the aluminium ion-containing compound is about 0.865.
33. The method of any one of clauses 1 to 32 wherein the concentration of the aluminium ion-containing compound is greater than 0.1 mg/ml.
34. The method of clause 33 wherein the concentration of the aluminium ion-containing compound is greater than 5 mg/ml.
35. The method of clause 34 wherein the concentration of the aluminium ion-containing compound is greater than 30 mg/ml.
36 The method of clause 35 wherein the concentration of the aluminium ion-containing compound is greater than 40.0 mg/ml.
37. The method of any one of clauses 1 to 36 wherein the concentration of the aluminium ion-containing compound is no greater than 1000 mg/ml.
38. The method of clause 37 wherein the concentration of the aluminium ion-containing compound is no greater than 100 mg/ml.
39. The method of clause 38 wherein the concentration of the aluminium ion-containing compound is no greater than 50 mg/ml.
40. The method of clause 39 wherein the concentration of the aluminium ion-containing compound is no greater than 41.0 mg/ml.
41. The method of any one of clauses 1 to 16 wherein the concentration of the aluminium ion-containing compound is 0.1 mg/ml to 1000 mg/ml.
42. The method of clause 41 wherein the concentration of the aluminium ion-containing compound is 5 mg/ml to 100 mg/ml.
43 The method of clause 42 wherein the concentration of the aluminium ion-containing compound is 30 mg/ml to 50 mg/ml.
44. The method of clause 43 wherein the concentration of the aluminium ion-containing compound is about 40.0 mg/ml.
45. The method of any one of clauses 1 to 44 wherein the concentration of the hydroxide ion-containing compound is greater than 0.1 mg/ml.
46. The method of clause 45 wherein the concentration of the hydroxide ion-containing compound is greater than 1 mg/ml.
47. The method of clause 46 wherein the concentration of the hydroxide ion-containing compound is greater than 10 mg/ml.
48 The method of clause 47 wherein the concentration of the hydroxide ion-containing compound is greater than 17.2 mg/ml.
49. The method of any one of clauses 1 to 48 wherein the concentration of the hydroxide ion-containing compound is no greater than 1000 mg/ml.
50 The method of clause 49 wherein the concentration of the hydroxide ion-containing compound is no greater than 80 mg/ml.
51. The method of clause 50 wherein the concentration of the hydroxide ion-containing compound is no greater than 25 mg/ml.
52 The method of clause 51 wherein the concentration of the hydroxide ion-containing compound is no greater than 18 mg/ml.
53. The method of any one of clauses 1 to 44 wherein the concentration of the hydroxide ion-containing compound is 0.1 mg/ml to 1000 mg/ml.
54. The method of clause 53 wherein the concentration of the hydroxide ion-containing compound is 1 mg/ml to 80 mg/ml.
55. The method of clause 54 wherein the concentration of the hydroxide ion-containing compound is 10 mg/ml to 25 mg/ml.
56. The method of clause 55 wherein the concentration of the hydroxide ion-containing compound is about 17.2 mg/ml.
57 The method of any one of clauses 1 to 56 wherein the mixing ratio of the aluminium ion-containing compound over the hydroxide ion-containing compound is 10/1 to 1/10.
58 The method of clause 57 wherein the mixing ratio of the aluminium ion-containing compound over the hydroxide ion-containing compound is 5/1 to 1/5.
59. The method of clause 58 wherein the mixing ratio of the aluminium ion-containing compound over the hydroxide ion-containing compound is 2/1 to 1/2.
60. The method of clause 59 wherein the mixing ratio of the aluminium ion-containing compound over the hydroxide ion-containing compound is 1.5/1 to 1/1.5.
61. The method of clause 60 wherein the mixing ratio of the aluminium ion-containing compound over the hydroxide ion-containing compound is about 1/1.
62. The method of any one of clauses 1 to 61 wherein the flow rate of the aluminium ion-containing compound is 1 ml/min to 100 ml/min.
63. The method of clause 62 wherein the flow rate of the aluminium ion-containing compound is 10 ml/min to 30 ml/min.
64. The method of clause 63 wherein the flow rate of the aluminium ion-containing compound is 15 ml/min to 25 ml/min.
65. The method of clause 64 wherein the flow rate of the aluminium ion-containing compound is about 20 ml/min.
66 The method of any one of clauses 1 to 65 wherein the flow rate of the hydroxide ion-containing compound is 1 ml/min to 100 ml/min.
67 The method of clause 66 wherein the flow rate of the hydroxide ion-containing compound is 10 ml/min to 30 ml/min.
68 The method of clause 67 wherein the flow rate of the hydroxide ion-containing compound is 15 ml/min to 25 ml/min.
69. The method of clause 68 wherein the flow rate of the hydroxide ion-containing compound is about 20 ml/min.
70. The method of any one of clauses 1 to 69 wherein the aluminium ion-containing compound and hydroxide ion-containing compound are reacted in a reactor having a volume of 0.1 to 1 ml.
71 The method of clause 70 wherein the aluminium ion-containing compound and hydroxide ion-containing compound are reacted in a reactor having a volume of 0.4 to 0.6 ml.
72. The method of clause 71 wherein the aluminium ion-containing compound and hydroxide ion-containing compound are reacted in a reactor having a volume of about 0.5 ml.
73. The method of any one of clauses 1 to 72 wherein the aluminium ion-containing compound and hydroxide ion-containing compound are mixed with a T-mixer.
74. The method of any one of clauses 1 to 73 wherein the composition is dialysed.
75 The method of clause 74 wherein the composition is dialysed to reduce the concentration of any unreacted aluminium ion-containing compound.
76. The method of clause 75 wherein the composition is dialysed to remove substantially all unreacted aluminium ion-containing compound.
77. The method of any one of clauses 74 to 76 wherein the dialysis membrane has a molecular cut-off of 2.5 to 4.5 KD.
78. The method of clause 77 wherein the dialysis membrane has a molecular cut-off of about 3.5 KD.
79 The method of any one of clauses 74 to 78 wherein the dialysis is performed using a citric acid buffer as a dialysis medium.
80 The method of clause 79 wherein the citric acid buffer has a concentration of 5 to 50 mM.
81. The method of clause 80 wherein the citric acid buffer has a concentration of 10 to 30 mM.
82. The method of clause 81 wherein the citric acid buffer has a concentration of about 20 mM.
83. The method of any one of clauses 79 to 82 wherein the citric acid buffer has a pH of 4.0 to 6.9.
84. The method of clause 83 wherein the citric acid buffer has a pH of 5.3 to 6.7.
85 The method of clause 84 wherein the citric acid buffer has a pH of 5.5 to 6.5.
86. The method of any one of clauses 79 to 85 wherein the citric acid buffer comprises a compound providing a counter-ion having a pKa of 2 to 3.
87. The method of clause 86 wherein the citric acid buffer comprises a compound providing a counter-ion having a pKa of about 2.5.
88. The method of any one of clauses 79 to 87 wherein the compound providing the counter-ion in the citric acid buffer is selected from the group consisting of lithium citrate, sodium citrate, potassium citrate and rubidium citrate; and hydrates thereof.
89. The method of clause 88 wherein the compound providing the counter-ion in the citric acid buffer is selected from the group consisting of sodium citrate, potassium citrate and hydrates thereof.
90. The method of clause 88 wherein the compound providing the counter-ion in the citric acid buffer is selected from the group consisting of sodium citrate and hydrates thereof.
91. The method of clause 88 wherein the compound providing the counter-ion in the citric acid buffer is sodium citrate dihydrate.
92. The method of any one of clauses 1 to 85 wherein the volume distribution mean diameter of the particles is 1.000 um or lower.
93. The method of clause 92 wherein the volume distribution mean diameter of the particles is 0.500 um or lower.
94. The method of clause 93 wherein the volume distribution mean diameter of the particles is 0.200 um or lower.
95. The method of clause 94 wherein the volume distribution mean diameter of the particles is 0.100 um or lower.
96. The method of any one of clauses 1 to 95 wherein the number distribution mean diameter of the particles is 1.000 um or lower.
97. The method of clause 96 wherein the number distribution mean diameter of the particles is 0.500 um or lower.
98. The method of clause 97 wherein the number distribution mean diameter of the particles is 0.200 um or lower.
99. The method of clause 98 wherein the number distribution mean diameter of the particles is 0.100 um or lower.
100. The method of any one of clauses 1 to 99 wherein the composition is filtered.
101. The method of clause 100 wherein the composition is filtered after dialysis.
102. The method of either clause 100 or 101 wherein the composition is filtered with a 0.45 um filter.
103. The method of any one of clauses 100 to 102 wherein the composition is filtered with a 0.22 um filter.
104. The method of any one of clauses 100 to 103 wherein the volume distribution mean diameter of the particles after filtration is 1.000 um or lower.
105. The method of clause 104 wherein the volume distribution mean diameter of the particles after filtration is 0.500 um or lower.
106. The method of clause 105 wherein the volume distribution mean diameter of the particles after filtration is 0.200 um or lower.
107. The method of clause 106 wherein the volume distribution mean diameter of the particles after filtration is 0.100 um or lower.
108. The method of any one of clauses 100 to 107 wherein the number distribution mean diameter of the particles after filtration is 1.000 um or lower.
109. The method of clause 108 wherein the number distribution mean diameter of the particles after filtration is 0.500 um or lower.
110. The method of clause 109 wherein the number distribution mean diameter of the particles after filtration is 0.200 um or lower.
111. The method of clause 110 wherein the number distribution mean diameter of the particles after filtration is 0.100 um or lower.
112. The method of any one of clauses 1 to 111 wherein the composition has a pH of 6.0 to 7.5.
113. The method of clause 112 wherein the composition has a pH of 6.5 to 7.0.
114. The method of any one of clauses 1 to 113 wherein the tonicity of the composition is adjusted.
115. The method of clause 114 wherein the tonicity of the composition is adjusted with sucrose.
116. The method of clause 115 wherein the concentration of the sucrose is 4 to 12% w/V.
117. The method of clause 116 wherein the concentration of the sucrose is 6 to 10% w/V.
118. The method of clause 117 wherein the concentration of the sucrose is about 8% w/V.
119. The method of any one of clauses 1 to 118 wherein a buffer is added to the composition.
120. The method of clause 119 wherein a histidine buffer is added to the composition.
121. The method of clause 120 wherein the concentration of the histidine buffer is 5 to 15 mM.
122. The method of clause 121 wherein the concentration of the histidine buffer is 7 to 13 mM.
123. The method of clause 122 wherein the concentration of the histidine buffer is about 10 mM.
124. The method of any one of clauses 119 to 123 wherein the histidine buffer has a pH of 6 to 7.
125. The method of clause 124 wherein the histidine buffer has a pH of about 6.5.
126. The method of any one of clauses 1 to 125 wherein the composition is made in a continuous flow process.
127. The method of any one of clauses 1 to 126 wherein no further constituents are added before the reaction.
128. A composition obtainable, such as obtained, by the method of any one of clauses 1 to 127.
129. An immunogenic composition comprising aluminium hydroxide particles obtained by the method of any one of clauses 1 to 127.
130. A composition comprising aluminium hydroxide particles and citric acid buffer.
131. An aqueous suspension comprising aluminium hydroxide particles and citric acid buffer.
132. The composition or suspension of either clause 130 or 131 wherein the aluminium hydroxide particles are coated in the citric acid buffer.
133. The composition or suspension of any one of clauses 130 to 132 wherein the citric acid buffer comprises citric acid and sodium citrate dihydrate.
134. The composition or suspension of any one of clauses 130 to 133 wherein the aluminium hydroxide particles are as defined in any one of clauses 1 to 128.
135. The composition or suspension of any one of clauses 130 to 134 further comprising aluminium chloride.
136. Use of an MF system to prepare a composition comprising aluminium hydroxide particles.
137. The method, composition or suspension of any one of clauses 1-135, wherein the composition or suspension is administered in an effective amount to a human subject.

### EXAMPLES

### Example 1: General Details of Mixing Apparatus

In the following examples, all samples were prepared by injecting NaOH and AlCl₃ solutions simultaneously via stainless steel syringes into a T-junction, after which the liquid then passes into a 0.5 mL reactor. The product is then collected from the reactor outlet. The mixing apparatus used in all experiments was the micro-fluidic system described in WO2019/2150022, FIG. 1.

All references in the examples below to ‘citric acid buffer’ or ‘citrate buffer’ refer to sodium citrate dihydrate.

The examples below refer to a ‘loading factor’. The loading factor is generally linearly correlated with the concentration of aluminium salt particles present in the solution. It was considered that a loading factor of less than 0.1 represented an insufficient presence of aluminium hydroxide particles for practical use (particularly in an adjuvant context), whereas a loading factor of greater than or equal to 0.1 represented a sufficient concentration of aluminium hydroxide particles.

Blank cells or cells containing a hyphen in the tables provided below contain the same data as the cell above unless otherwise indicated. ‘NR’ stands for ‘no result’. In ‘filtration’, ‘dialysis’, ‘PS20’ and ‘dialysis buffer’ columns, a blank cell indicates ‘no’ and a cell containing ‘x’ indicates ‘yes’.

### Example 2: Molar Ratio and Mixing Ratio of AlCl₃ and NaOH

Different molar ratios and mixing ratios of NaOH/AlCl₃ were investigated, firstly using 40 mg/mL AlCl₃·6H₂O and then using 20 mg/mL AlCl₃·6H₂O. For every test, a different NaOH solution was prepared and added to a stock solution of AlCl₃. These stock solutions were prepared in quantities of 500 mL. Of each combination of molar ratio and mixing ratio, 4 different flow rates were applied between 5 and 30 mL/min. The pH was measured using a titration instrument and calibrated pH probe for water analysis between pH 1 and 13. To perform titrations, a stock solution of 1N HCl was prepared, as well as a citric acid buffer. This buffer was set at a pH of 3 at a molarity of 1M. While the product is stirred vigorously, the acid or acid buffer was added dropwise until the desired pH was reached. Then the solution was further stirred for 5 minutes before the end value was noted. Sample DLS results were collected using an accumulation of 5 repeated exposures of 60 s to light.

The matrix of parameters which were tested is set out in Table 1 below.

TABLE 1

| AlCl₃•6H₂O | | Flow rate | | |
|---|---|---|---|---|
| conc. | NaOH | AlCl₃•6H₂O | | |
| mg/mL | M | mL/min | Filtration | pH adjustment |
| 20 | 0.5 | 5 | None | None |
| 40 | 1 | 10 | Prior to pH | Neutral pH (HCl) |
| | | | adjustment | |
| | 1.5 | 20 | After pH | pH = 5 (HCl) |
| | | | adjustment | |
| | 3 | 40 | | Neutral pH (citric |
| | | | | acid buffer) |
| | | | | pH = 5 (citric |
| | | | | acid buffer) |

Experiments Using 40 mg/mL AlCl₃·6H₂O

Details of the samples tested are provided in Table 2a below.

TABLE 2a

| | | Molar | Mixing | |
|---|---|---|---|---|
| | | ratio | ratio | |
| | Sample | NaOH/ | AlCl₃/ | |
| Test series | number | AlCl₃ | NaOH | Further details |
| ALU.BRI.001 | 1-12 | 1.0 | 1/1 | pH adjustment using citrate |
| | | | | buffer |
| | 13-24 | 0.5 | 2/1 | pH adjustment using citrate |
| | | | | buffer |
| ALU.BRI.002 | 1-12 | 1.0 | 2/1 | pH adjustment using citrate |
| | | | | buffer |
| | 13-24 | 1.0 | 2/1 | Added citrate buffer to |
| | | | | AlCl₃ solution prior to |
| | | | | nanoparticle production |
| ALU.BRI.003 | 1-12 | 1.0 | 2/1 | pH adjustment using HCl |
| | 13-24 | 1.5 | 2/1 | pH adjustment using HCl |
| | 25-36 | 1.5 | 2/1 | pH adjustment using citrate |
| | | | | buffer |
| ALU.BRI.004 | 1-12 | 1.26 | 2/1 | pH adjustment using HCl |
| | 13-24 | 1.26 | 2/1 | pH adjustment using citrate |
| | | | | buffer |
| ALU.BRI.005 | 1-12 | 0.63 | 4/1 | pH adjustment using citrate |
| | | | | buffer |
| | 13-24 | 1.0 | 4/1 | pH adjustment using citrate |
| | | | | buffer |
| ALU.BRI.006 | 1-12 | 1.5 | 4/1 | pH adjustment using using |
| | | | | citrate buffer |
| | 13-24 | 1.5 | 1/1 | pH adjustment using citrate |
| | | | | buffer |
| | 25-36 | 1.26 | 1/1 | pH adjustment using citrate |
| | | | | buffer |
| ALU.BRI.007 | 1-12 | 1.5 | 1/2 | pH adjustment using citrate |
| | | | | buffer |

The results of these experiments are set out in Tables 2b to 2 h below.

The first test series ALU.BRI.001 samples 1-12 used a mixing ratio of 1/1 and molar ratio of 1.0. A flow rate of 5 mL/min was found insufficient to obtain full reaction, hence the pH stayed at the value of 3.2. The other tests resulted in slightly basic conditions, with high values for the average volume-based particle size Mv above 2. Loading factors were generally acceptable.

In test series ALU.BRI.001 samples 13-24, an off stochiometric mixture was created using a mixing ratio of 2/1 and molar ratio of 0.5. As a result, the pH values remained low, as not all AlCl₃ was reacted out of the medium. Although generally the loading factors were quite low, very small particles are obtained. To samples 16, 19 and 22 some acetic acid buffer was added to check the samples' behavior in response (samples 17, 20, 23). To a part of sample 22 NaOH was added resulting in sample 24. The remaining AlCl₃ reacted with NaOH and flocculated heavily without changes to the overall acidity. This resulted in a clear bimodal signal of the DLS: about 30% in volume of the particles were smaller than 100 nm, the remainder of the particles were larger than 1 μm. However, for these samples the general DLS loading was too low prior to filtration.

In test series ALU.BRI.002, the same molar excess of 1.0 was tested using the facility of slightly adjusting the pH with buffer. Samples 3, 6, 9, 12, 15, 18, 21 and 24 were created by adding 1M NaOH solution to reach a pH value of about 7. Generally acceptable loading factors are obtained.

In test series ALU.BRI.003 samples 1 to 12 a comparison in identical mixing ratios was made with ALU.BRI.002.

In general, untreated samples had pH values around 9 when a stoichiometric excess (NaOH/AlCl₃) of 1 was used. Using stoichiometric excesses of 1.5, the resulting pH increased to 10 to 11, which had an impact on the overall DLS loading. When adding HCl to adjust the pH (ALU.BRI.003 samples 1-12) the Al(OH)₃ precipitated from solution yielding increased DLS loading values to acceptable ranges, but also drastically increasing particle sizes. Adding citrate had an inverse effect, lowering the loading values even more but resulting in very low particle sizes, indicating an even further dissolution of Al(OH)₃ as shown in ALU.BRI.003 samples 25-36.

In ALU.BRI.004 the molar ratio of 1.25 and mixing ratio of 2/1 resulted in high pH for the neat samples and therefore low loading of the DLS sample. This effect was reduced by adding both the HCl and citrate buffers leading to more acceptable loading results.

In ALU.BRI.005 samples 1 to 12, lower molar stoichiometries were tested, rendering a slightly acidic end product. Samples 2, 5, 8, 11 were treated with citric acid buffer. At low flow rates an incomplete reaction was observed, rendering low loading. Higher flow rates rendered a bimodal or trimodal DLS signal. Samples were milky white and unfilterable.

For ALU.BRI.005 samples 13 to 24 a stoichiometric excess of 1.0 was set at mixing ratio 4/1. High pH values were obtained resulting in low loading measured in the DLS experiments. Generally, these samples contained a high portion of large particle sizes (volume average) such that filtration was not feasible for these samples. It may be concluded that a mixing ratio of 4/1 (or higher) is not optimal for small particle production.

In sample series ALU.BRI.006 the last stoichiometric condition was tested for mixing ratio 4/1, as well as molar ratios 1.25 and 1.5 with mixing ratio 1/1. Generally the loading factors for the molar ratios higher than 1 were too low, indicating dissolution of the product. Overall the 4/1 mixing ratio does not produce desirable particle characteristics (samples 13-24 of the ALU.BRI.005 test series), or produces a low loading factor when a stoichiometric excess of 1.5 is used.

Comparing the results of mixing ratio 1/1 and different molar ratios (ALU.BRI.001 samples 1-12 and ALU.BRI.006 samples 13-36) similar outcomes can be noted: both the molar ratios of 1.5 and 1.25 rendered low loadings, and the molar ratio of 1.0 (ALU.BRI.001) produced filterable material if the resulting pH right after formation was not too high.

Finally, a mixing ratio of ½ and molar ratio of 1.5 was tested in the series ALU.BRI.007 samples 1-12. As with all tests with higher stoichiometric excess, low DLS loadings were retrieved.
In the tables below the headings ‘pH’, ‘pH=7’ and ‘pH=5’ below citrate refer to the following: ‘pH’=pH without adjustment, ‘pH=7’=adjusted to pH 7 using citrate and ‘pH=5’=adjusted to pH 5 using citrate.

TABLE 2b

| ALU.BRI.001 | | | | citrate buffer | | | |
|---|---|---|---|---|---|---|---|
| QAlCl3 | QNaOH | sample | | pH ad- | Mv | Mn | DLS |
| mL/min | mL/min | number | pH | justed | μm | μm | loading |
| 5 | 5 | 1 | 3.2 | | 2.7 | 0.0702 | 0.132 |
| | | 2 | | | NR | NR | NR |
| | | 3 | | | NR | NR | NR |
| 10 | 10 | 4 | 9.65 | | 2.9 | 1.003 | 0.172 |
| | | 5 | | 6.94 | 2.357 | 0.543 | 0.358 |
| | | 6 | | 5.52 | 1.43 | 0.1293 | 0.393 |
| 20 | 20 | 7 | 9.17 | | 3.13 | 0.0221 | 0.034 |
| | | 8 | | 7.16 | 3.18 | 0.583 | 0.358 |
| | | 9 | | 4.94 | 4.48 | 0.362 | 0.96 |
| 30 | 30 | 10 | 8.84 | | 5.93 | 3.29 | 0.00826 |
| | | 11 | | 7.28 | 0.705 | 0.0682 | 0.142 |
| | | 12 | | 4.92 | 2.98 | 0.157 | 0.54 |
| 5 | 2.5 | 13 | 3.72 | | 5 | 0.0829 | 3.93 |
| | | 14 | | | NR | NR | NR |
| | | 15 | | | NR | NR | NR |
| 10 | 5 | 16 | 3.73 | | 0.961 | 0.0429 | 0.106 |
| | | 17 | | 2.95 | 0.0708 | 0.0649 | 0.0391 |
| | | 18 | | | NR | NR | NR |
| 20 | 10 | 19 | 3.78 | | 1.27 | 0.2137 | 0.0393 |
| | | 20 | | 2.58 | 0.02692 | 0.00358 | 0.0346 |
| | | 21 | | | NR | NR | NR |
| 30 | 15 | 22 | 4.26 | | 0.964 | 0.01152 | 0.00746 |
| | | 23 | | 3.32 | 0.1263 | 0.01099 | 0.00935 |
| | | 24 | | 4.2 | 1.998 | 0.01287 | 0.114 |

TABLE 2c
ALU.BRI.002

| | | | pH ad- | | | | | |
|---|---|---|---|---|---|---|---|---|
| QAlCl3 | QNaOH | sample | justed | | pH ad- | Mv | Mn | DLS |
| mL/min | mL/min | number | before | pH | justed | μm | μm | loading |
| 5 | 2.5 | 1 | no | 4.3 | | 2.331 | 0.1506 | 3.3 |
| | | 2 | no | | 3.08 | 2.778 | 0.1095 | 3.51 |
| | | 3 | no | | 9.32 | 1.364 | 1.331 | 2.9 |
| 10 | 5 | 4 | no | 4.39 | | 1.819 | 0.0824 | 2.25 |
| | | 5 | no | | 3.2 | 2.167 | 0.058 | 1.41 |
| | | 6 | no | | 6.5 | 1.496 | 1.434 | 0.266 |
| 20 | 10 | 7 | no | 4.33 | | 1.107 | 0.0558 | 4.69 |
| | | 8 | no | | 2.86 | 1.245 | 0.098 | 1.4 |
| | | 9 | no | | 7.6 | 0.86 | 0.2216 | 1.1 |
| 30 | 15 | 10 | no | 4.44 | | 1.559 | 0.0611 | 1.77 |
| | | 11 | no | | 2.96 | 0.332 | 0.0204 | 0.476 |
| | | 12 | no | | 7.02 | 1.341 | 1.312 | 0.0498 |
| 5 | 2.5 | 13 | yes | 4.32 | | 2.436 | 0.1352 | 4.56 |
| | | 14 | yes | | 3.1 | 2.12 | 0.1886 | 2.62 |
| | | 15 | yes | | 8.65 | 1.523 | 0.496 | 1.6 |
| 10 | 5 | 16 | yes | 4.3 | | 2.121 | 0.1813 | 2.15 |
| | | 17 | yes | | 3.04 | 0.425 | 0.1055 | 0.0155 |
| | | 18 | yes | | 7.8 | 3.12 | 10.89 | 4.72 |
| 20 | 10 | 19 | yes | 4.11 | | 0.68 | 0.0591 | 0.282 |
| | | 20 | yes | | 2.85 | 0.2844 | 0.1174 | 0.00799 |
| | | 21 | yes | | 8.1 | 1.778 | 0.798 | 6.28 |
| 30 | 15 | 22 | yes | 4.4 | | 1.705 | 0.0855 | 1.71 |
| | | 23 | yes | | 2.8 | 1.363 | 0.0342 | 0.0282 |
| | | 24 | yes | | 7.4 | 2.37 | 0.449 | 1.51 |

TABLE 2d

| ALU.BRI.003 | | | | | | | |
|---|---|---|---|---|---|---|---|
| AlCl₃•6H₂O | NaOH | AlCl₃•6H₂O | NaOH | | | | |
| conc | conc | conc | conc | mixing | | molar | QAlCl₃ |
| mg/mL | mg/mL | mol/ml | mol/ml | ratio | | excess | mL/min |
| 40 | 40 | 0.000165679 | 0.001 | 2 | 1 | 1.005958333 | 5 |
| | | | | | | | 10 |
| | | | | | | | 20 |
| | | | | | | | 30 |
| 40 | 60 | 0.000165679 | 0.0015 | 2 | 1 | 1.5089375 | 5 |
| | | | | | | | 10 |
| | | | | | | | 20 |
| | | | | | | | 30 |
| 40 | 60 | 0.000165679 | 0.0015 | 2 | 1 | 1.5089375 | 5 |
| | | | | | | | 10 |
| | | | | | | | 20 |
| | | | | | | | 30 |

| | | | | | | DLS |
|---|---|---|---|---|---|---|
| | | | ph ad- | | | loading |
| QNaOH | sample | | justed | Mv | Mn | DLS |
| mL/min | number | pH | HCl | μm | μm | loading |
| 2.5 | 1 | 9.35 | | 1.357 | 0.082 | 0.0598 |
| | 2 | | 5.96 | 1.276 | 0.178 | 0.153 |
| | 3 | | 4.55 | 1.668 | 0.249 | 0.915 |
| 5 | 4 | 9.84 | | 1.41 | 0.21 | 0.143 |
| | 5 | | 7.78 | 1.173 | 1.098 | 0.0104 |
| | 6 | | 5.45 | 1.914 | 0.368 | 0.348 |
| 10 | 7 | 9.68 | | 1.46 | 1.297 | 0.481 |
| | 8 | | 7.77 | 1.298 | 1.224 | 0.251 |
| | 9 | | 5.5 | 1.537 | 0.404 | 0.662 |
| 15 | 10 | 9.95 | | 1.244 | 0.167 | 0.0831 |
| | 11 | | 7.49 | 2.1 | 1.583 | 0.264 |
| | 12 | | 4.62 | 1.351 | 0.346 | 2.67 |
| 2.5 | 13 | 10.24 | | 0.527 | 0.1094 | 0.0043 |
| | 14 | | 7.1 | 0.652 | 0.0936 | 0.16 |
| | 15 | | 4.5 | 2.385 | 0.309 | 2.6 |
| 5 | 16 | 11.5 | | 0.757 | 0.022 | 0.00135 |
| | 17 | | 6.92 | 2.297 | 0.385 | 4.17 |
| | 18 | | 4.12 | 2.766 | 0.1788 | 6.46 |
| 10 | 19 | 11.118 | | 0.713 | 0.0603 | 0.0075 |
| | 20 | | 7.2 | 1.249 | 1.223 | 3.74 |
| | 21 | | 4.6 | 1.466 | 1.432 | 0.543 |
| 15 | 22 | 11.01 | | 0.57 | 0.0432 | 0.000716 |
| | 23 | | 7.4 | 1.325 | 1.229 | 1.13 |
| | 24 | | 4.4 | 2.345 | 0.2779 | 7 |
| | | | Citrate buffer | | | |
| 2.5 | 25 | | | NR | NR | NR |
| | 26 | | 6.76 | 1.509 | 0.0742 | 0.0668 |
| | 27 | | 4.95 | 0.0481 | 0.00091 | 0.0021 |
| 5 | 28 | | | NR | NR | NR |
| | 29 | | 7.06 | 0.00226 | 0.00443 | 0.00826 |
| | 30 | | 4.9 | 0.41 | 0.00375 | 0.52 |
| 10 | 31 | | | NR | NR | NR |
| | 32 | | 6.98 | 0.0035 | 0.00264 | 0.00202 |
| | 33 | | 5.1 | 0.00636 | 0.00944 | 0.000613 |
| 15 | 34 | | | NR | NR | NR |
| | 35 | | 5.71 | 0.607 | 0.00523 | 0.000926 |
| | 36 | | 4.43 | 1.281 | 0.0681 | 0.0373 |

TABLE 2e

| ALU.BRI.004 | | | | | | | | | | | | | | |
|---|---|---|---|---|---|---|---|---|---|---|---|---|---|---|
| AlCl₃•6H₂O | NaOH | AlCl₃•6H₂O | NaOH | | | | | | | | ph ad- | | | |
| conc | conc | conc | conc | mixing | | molar | QAlCl₃ | QNaOH | sample | | justed | Mv | Mn | DLS |
| mg/mL | mg/mL | mol/ml | mol/ml | ratio | | excess | mL/min | mL/min | number | pH | HCl | μm | μm | loading |
| 40 | 50 | 0.000165679 | 0.00125 | 2 | 1 | 1.257447917 | 5 | 2.5 | 1 | 11.6 | | 2.054 | 0.32 | 0.000673 |
| | | | | | | | | | 2 | | 7.42 | 5.69 | 0.655 | 0.00176 |
| | | | | | | | | | 3 | | 5.2 | 1.254 | 0.128 | 4.17 |
| | | | | | | | 10 | 5 | 4 | 11.05 | | 5.41 | 5.11 | 0.000956 |
| | | | | | | | | | 5 | | 7.2 | 4.45 | 0.214 | 0.0311 |
| | | | | | | | | | 6 | | 5.3 | 1.505 | 0.1084 | 2.71 |
| | | | | | | | 20 | 10 | 7 | 10.9 | | 0.861 | 0.315 | 0.00602 |
| | | | | | | | | | 8 | | 7.42 | 3.22 | 0.1919 | 0.0268 |
| | | | | | | | | | 9 | | 5.1 | 1.672 | 0.0847 | 3.17 |
| | | | | | | | 30 | 15 | 10 | 12.25 | | 4.47 | 1.873 | 0.000583 |
| | | | | | | | | | 11 | | 7 | 1.97 | 0.07 | 0.299 |
| | | | | | | | | | 12 | | 5.4 | 3.4 | 0.173 | 6.63 |
| | | | | | | | | | | | citrate buffer | | | |
| 40 | 50 | 0.000165679 | 0.00125 | 2 | 1 | 1.257447917 | 5 | 2.5 | 13 | | | NR | NR | NR |
| | | | | | | | | | 14 | | 7.16 | 1.65 | 0.2212 | 0.529 |
| | | | | | | | | | 15 | | 4.96 | 2.951 | 0.829 | 0.217 |
| | | | | | | | 10 | 5 | 16 | | | NR | NR | NR |
| | | | | | | | | | 17 | | 7.1 | 1.913 | 0.2319 | 0.294 |
| | | | | | | | | | 18 | | 5.13 | 1.988 | 0.406 | 0.0503 |
| | | | | | | | 20 | 10 | 19 | | | NR | NR | NR |
| | | | | | | | | | 20 | | 7.1 | 1.04 | 0.822 | 0.545 |
| | | | | | | | | | 21 | | 5.1 | 0.841 | 0.063 | 0.00118 |
| | | | | | | | 30 | 15 | 22 | | | NR | NR | NR |
| | | | | | | | | | 23 | | 6.7 | 3.12 | 1.182 | 0.536 |
| | | | | | | | | | 24 | | 4.6 | 2.44 | 0.363 | 0.938 |

TABLE 2f

| ALU.BRI.005 | | | | | | | |
|---|---|---|---|---|---|---|---|
| AlCl₃•6H₂O | NaOH | AlCl₃•6H₂O | NaOH | | | | |
| conc | conc | conc | conc | mixing | | molar | |
| mg/mL | mg/mL | mol/ml | mol/ml | ratio | | excess | Qalu |
| 40 | 50 | 0.000165679 | 0.00125 | 4 | 1 | 0.628723958 | 5 |
| | | | | | | | 10 |
| | | | | | | | 20 |
| | | | | | | | 30 |
| 40 | 80 | 0.000165679 | 0.002 | 4 | 1 | 1.005958333 | 5 |
| | | | | | | | 10 |
| | | | | | | | 20 |
| | | | | | | | 30 |

| | | | | | | | 1week | |
|---|---|---|---|---|---|---|---|---|
| | sample | | | citrate | MV | Mn | MV | Mn |
| QNaOH | number | pH | pH = 7 | pH = 5 | μm | μm | μm | μm |
| 1.25 | 1 | | 5 | | 0.149 | 0.0326 | 0.656 | 0.1231 |
| | 2 | | | 4 | 3.77 | 0.1014 | 0.1837 | 0.1786 |
| | 3 | | | | NR | NR | NR | NR |
| 2.5 | 4 | | 5.1 | | 2.824 | 0.0684 | 0.545 | 0.0566 |
| | 5 | | | 4.16 | 3.41 | 0.0569 | 0.0445 | 0.00672 |
| | 6 | | | | NR | NR | NR | NR |
| 5 | 7 | | 4.9 | | 2.661 | 0.077 | NR | NR |
| | 8 | | | 3.89 | 2.843 | 0.533 | NR | NR |
| | 9 | | | | NR | NR | NR | NR |
| 7.5 | 10 | | 5.1 | | 2.433 | 0.0784 | NR | NR |
| | 11 | | | 3.7 | 1.197 | 0.0364 | NR | NR |
| | 12 | | | | NR | NR | NR | NR |
| 1.25 | 13 | 9.35 | | | 3.62 | 0.2787 | NR | NR |
| | 14 | | 6.7 | | 3.52 | 0.655 | NR | NR |
| | 15 | | | | NR | NR | NR | NR |
| 2.5 | 16 | 10.67 | | | 3.72 | 0.655 | NR | NR |
| | 17 | | 6.72 | | 3.83 | 0.391 | NR | NR |
| | 18 | | | 4.5 | 1.805 | 0.317 | NR | NR |
| 5 | 19 | 11.12 | | | 0.91 | 0.533 | NR | NR |
| | 20 | | 7.1 | | NR | NR | NR | NR |
| | 21 | | | 5.22 | 1.137 | 1.111 | NR | NR |
| 7.5 | 22 | 9.87 | | | 1.39 | 0.0877 | NR | NR |
| | 23 | | 5.8 | | 1.919 | 0.1531 | NR | NR |
| | 24 | | | 4.38 | 1.984 | 0.0808 | NR | NR |

TABLE 2g

| ALU.BRI.006 | | | | | | | | | | | | | | |
|---|---|---|---|---|---|---|---|---|---|---|---|---|---|---|
| AlCl₃•6H₂O | NaOH | AlCl₃•6H₂O | NaOH | | | | | | | | | | | |
| conc | conc | conc | conc | mixing | | molar | | | sample | | pH = | citrate | MV | Mn |
| mg/mL | mg/mL | mol/ml | mol/ml | ratio | | excess | Qalu | QNaOH | number | pH | 7 | pH = 5 | μm | μm |
| 40 | 120 | 0.000165679 | 0.003 | 4 | 1 | 1.5089375 | 5 | 1.25 | 1 | 12.2 | | | 0.885 | 0.719 |
| | | | | | | | | | 2 | 6.85 | | | 1.542 | 0.927 |
| | | | | | | | | | 3 | 5.22 | | | 1.142 | 0.927 |
| | | | | | | | 10 | 2.5 | 4 | 13.6 | | | 1.43 | 0.863 |
| | | | | | | | | | 5 | 7.3 | | | 0.709 | 0.41 |
| | | | | | | | | | 6 | 5.39 | | | 0.619 | 0.506 |
| | | | | | | | 20 | 5 | 7 | 12.7 | | | 0.709 | 0.278 |
| | | | | | | | | | 8 | 7.2 | | | NR | NR |
| | | | | | | | | | 9 | 5.21 | | | 0.801 | 0.569 |
| | | | | | | | 30 | 7.5 | 10 | 11.8 | | | 0.794 | 0.614 |
| | | | | | | | | | 11 | 7.42 | | | NR | NR |
| | | | | | | | | | 12 | 5.2 | | | 0.744 | 0.509 |
| 40 | 30 | 0.000165679 | 0.00075 | 1 | 1 | 1.5089375 | 5 | 5 | 13 | 11.2 | | | 0.754 | 0.1988 |
| | | | | | | | | | 14 | 7.23 | | | NR | NR |
| | | | | | | | | | 15 | 5.1 | | | 0.368 | 0.0491 |
| | | | | | | | 10 | 10 | 16 | 11.36 | | | 0.749 | 0.641 |
| | | | | | | | | | 17 | 6.73 | | | 0.849 | 0.305 |
| | | | | | | | | | 18 | 5.13 | | | 0.471 | 0.0608 |
| | | | | | | | 20 | 20 | 19 | 12.15 | | | 0.273 | 0.0774 |
| | | | | | | | | | 20 | 7.2 | | | 0.478 | 0.163 |
| | | | | | | | | | 21 | 5.12 | | | 0.506 | 0.182 |
| | | | | | | | 30 | 30 | 22 | 12.13 | | | 0.592 | 0.115 |
| | | | | | | | | | 23 | 7.13 | | | 0.304 | 0.1153 |
| | | | | | | | | | 24 | 5.23 | | | 0.388 | 0.248 |
| 40 | 25 | 0.000165679 | 0.000625 | 1 | 1 | 1.257447917 | 5 | 5 | 25 | 11.1 | | | NR | NR |
| | | | | | | | | | 26 | 7.07 | | | 1.62 | 0.1834 |
| | | | | | | | | | 27 | 4.6 | | | 2.98 | 0.385 |
| | | | | | | | 10 | 10 | 28 | 11.22 | | | 1.185 | 0.0448 |
| | | | | | | | | | 29 | 7.2 | | | 0.612 | 0.1504 |
| | | | | | | | | | 30 | 5.11 | | | 2.132 | 0.1775 |
| | | | | | | | 20 | 20 | 31 | 11.2 | | | 0.387 | 0.2372 |
| | | | | | | | | | 32 | 6.64 | | | 1.302 | 0.1062 |
| | | | | | | | | | 33 | 5.07 | | | 1.666 | 0.1732 |
| | | | | | | | 30 | 30 | 34 | 11.3 | | | 3.21 | 1.233 |
| | | | | | | | | | 35 | 6.2 | | | 0.32 | 0.213 |
| | | | | | | | | | 36 | 5.1 | | | 2.1 | 0.836 |

TABLE 2h

| ALU.BRI.007 | | | | | | | | | | | | | | |
|---|---|---|---|---|---|---|---|---|---|---|---|---|---|---|
| AlCl₃•6H₂O | NaOH | AlCl₃•6H₂O | NaOH | | | | | | | | | | | |
| conc | conc | conc | conc | mixing | | molar | | | sample | | pH = | citrate | MV | Mn |
| mg/mL | mg/mL | mol/ml | mol/ml | ratio | | excess | Qalu | QNaOH | number | pH | 7 | pH = 5 | μm | μm |
| 40 | 15 | 0.000165679 | 0.000375 | 1 | 2 | 1.5089375 | 2.5 | 5 | 1 | 12.1 | | | 0.667 | 0.35 |
| | | | | | | | | | 2 | 7.5 | | | 0.00319 | 0.0099 |
| | | | | | | | | | 3 | 4.74 | | | 0.537 | 0.1047 |
| | | | | | | | 5 | 10 | 4 | 12.24 | | | 0.963 | 0.1095 |
| | | | | | | | | | 5 | 7.3 | | | 0.00274 | 0.00098 |
| | | | | | | | | | 6 | 5.1 | | | 0.493 | 0.00927 |
| | | | | | | | 10 | 20 | 7 | 11.4 | | | 0.53 | 0.1284 |
| | | | | | | | | | 8 | 6.96 | | | 0.397 | 0.00455 |
| | | | | | | | | | 9 | 5.1 | | | 0.348 | 0.0856 |
| | | | | | | | 20 | 40 | 10 | 11.25 | | | 0.1588 | 0.0424 |
| | | | | | | | | | 11 | 7.35 | | | 0.00729 | 0.00686 |
| | | | | | | | | | 12 | 5.1 | | | 0.325 | 0.1399 |

Experiments Using 20 mg/mL AlCl₃·6H₂O

For these experiments, the 4/1 mixing ratio was not used. The matrix of parameters which were tested is set out in Table 3a below.

TABLE 3a

| | | Molar | Mixing | |
|---|---|---|---|---|
| | | ratio | ratio | |
| | Sample | NaOH/ | AlCl₃/ | |
| Test series | number | AlCl₃ | NaOH | Further details |
| ALU.BRI.008 | 1-12 | 1.5 | 1/1 | pH adjustment using citrate |
| | | | | buffer |
| | 13-24 | 1.26 | 1/1 | pH adjustment using citrate |
| | | | | buffer |
| | 25-36 | 1.0 | 1/1 | pH adjustment using citrate |
| | | | | buffer |
| ALU.BRI.009 | 1-12 | 1.0 | 2/1 | pH adjustment using citrate |
| | | | | buffer |
| | 13-24 | 1.26 | 2/1 | pH adjustment using citrate |
| | | | | buffer |
| | 25-36 | 1.5 | 2/1 | pH adjustment using citrate |
| | | | | buffer |
| ALU.BRI.010 | 1-12 | 1.0 | 1/2 | pH adjustment using citrate |
| | | | | buffer |
| | 13-24 | 1.26 | 1/2 | pH adjustment using citrate |
| | | | | buffer |
| | 25-36 | 1.5 | 1/2 | pH adjustment using citrate |
| | | | | buffer |

The results of these experiments are set out in Tables 3b to 3d below.

TABLE 3b

| ALU.BRI.008 | | | | | | |
|---|---|---|---|---|---|---|
| AlCl₃•6H₂O | NaOH | AlCl₃•6H₂O | NaOH | | | |
| conc | conc | conc | conc | mixing | | molar |
| mg/mL | mg/mL | mol/ml | mol/ml | ratio | | excess |
| 20 | 15 | 8.28397E−05 | 0.000375 | 1 | 1 | 1.5089375 |
| 20 | 12.5 | 8.28397E−05 | 0.000313 | 1 | 1 | 1.257447917 |
| 20 | 10 | 8.28397E−05 | 0.00025 | 1 | 1 | 1.005958333 |

| | | sample | | pH = | citrate | MV | Mn | |
|---|---|---|---|---|---|---|---|---|
| Qalu | QNaOH | number | pH | 7 | pH = 5 | μm | μm | loading |
| 5 | 5 | 1 | 10.7 | | | 0.709 | 0.0601 | 0.00218 |
| | | 2 | 4.8 | | | NR | NR | NR |
| | | 3 | 3.9 | | | 0.27 | | 0.0000831 |
| 10 | 10 | 4 | 10.55 | | | 0.1438 | 0.0543 | 0.00725 |
| | | 5 | 6.6 | | | 0.669 | 0.1732 | 0.00393 |
| | | 6 | 4.95 | | | 0.308 | 0.1182 | 0.00689 |
| 20 | 20 | 7 | 11.97 | | | 0.0769 | 0.0499 | 0.000757 |
| | | 8 | 7.25 | | | 0.1111 | 0.02238 | 0.000994 |
| | | 9 | 4.98 | | | 0.1365 | 0.0659 | 0.0105 |
| 30 | 30 | 10 | 12 | | | 0.2156 | 0.0899 | 0.0000388 |
| | | 11 | 6.8 | | | 0.1645 | 0.0667 | 0.025 |
| | | 12 | 4.64 | | | 1.399 | 0.175 | 0.00000945 |
| 5 | 5 | 13 | 11.5 | | | 0.847 | 0.2446 | 0.00328 |
| | | 14 | 7 | | | 0.523 | 0.1149 | 0.0348 |
| | | 15 | 5.35 | | | 0.354 | NR | 0.00537 |
| 10 | 10 | 16 | 4.38 | | | 0.1413 | 0.031 | 0.286 |
| | | 17 | 3 | | | 0.0629 | 0.02936 | 0.168 |
| | | 18 | | | | NR | NR | NR |
| 20 | 20 | 19 | 4.38 | | | 0.026 | 0.0097 | 0.0054 |
| | | 20 | 3 | | | 0.2582 | 0.146 | 0.000263 |
| | | 21 | | | | NR | NR | NR |
| 30 | 30 | 22 | 4.3 | | | 0.01264 | 0.01088 | 0.00438 |
| | | 23 | 2.86 | | | 0.524 | 0.1036 | 0.00135 |
| | | 24 | | | | NR | NR | NR |
| 5 | 5 | 25 | 9.04 | | | 0.879 | 0.0472 | 0.0587 |
| | | 26 | 5.35 | | | 2.31 | NR | 0.0563 |
| | | 27 | 4 | | | 2.885 | NR | 0.0275 |
| 10 | 10 | 28 | 9.63 | | | 4.53 | 3.65 | 0.000809 |
| | | 29 | 7.36 | | | 2.44 | 0.625 | 0.0144 |
| | | 30 | 4.5 | | | 3.99 | 0.2596 | 0.173 |
| 20 | 20 | 31 | 9.2 | | | 3.28 | 0.41 | 0.00873 |
| | | 32 | 7.32 | | | 3.29 | 0.598 | 0.00739 |
| | | 33 | 4.7 | | | 2.611 | 1.863 | 0.00671 |
| 30 | 30 | 34 | 9.8 | | | 1.087 | NR | 0.000824 |
| | | 35 | 7.3 | | | 0.537 | 0.0983 | 0.00463 |
| | | 36 | 4.9 | | | 2.224 | 0.0409 | 0.0125 |

TABLE 3c

| ALU.BRI.009 | | | | | | |
|---|---|---|---|---|---|---|
| AlCl₃•6H₂O | NaOH | AlCl₃•6H₂O | NaOH | | | |
| conc | conc | conc | conc | mixing | | molar |
| mg/mL | mg/mL | mol/ml | mol/ml | ratio | | excess |
| 20 | 20 | 8.28397E−05 | 0.0005 | 2 | 1 | 1.005958333 |
| 20 | 25 | 8.28397E−05 | 0.000625 | 2 | 1 | 1.257447917 |
| 20 | 30 | 8.28397E−05 | 0.00075 | 2 | 1 | 1.5089375 |

| | | sample | | pH = | citrate | MV | Mn | |
|---|---|---|---|---|---|---|---|---|
| Qalu | QNaOH | number | pH | 7 | pH = 5 | μm | μm | loading |
| 5 | 2.5 | 1 | 10.4 | | | 3.16 | 0.623 | 0.025 |
| | | 2 | 7.3 | | | 1.63 | 1.354 | 1.27 |
| | | 3 | 4.3 | | | 2.538 | 1.595 | 0.799 |
| 10 | 5 | 4 | 10.3 | | | 1.334 | 1.195 | 0.66 |
| | | 5 | 6.3 | | | 1.438 | 1.34 | 1.34 |
| | | 6 | 4.7 | | | 1.27 | 1.264 | 1.4 |
| 20 | 10 | 7 | 10.4 | | | 3.02 | 0.46 | 0.079 |
| | | 8 | 7.2 | | | 1.411 | 0.1292 | 0.371 |
| | | 9 | 4.6 | | | 1.147 | 1.119 | 0.436 |
| 30 | 15 | 10 | 10.4 | | | 1.458 | 0.701 | 0.435 |
| | | 11 | 6.6 | | | 1.169 | 1.138 | 0.434 |
| | | 12 | 4.8 | | | 1.798 | 1.466 | 0.167 |
| 5 | 2.5 | 13 | 11.26 | | | 2.762 | 0.1548 | 1.22 |
| | | 14 | 7.07 | | | 1.895 | 1.347 | 1.55 |
| | | 15 | 5.2 | | | 1.883 | 0.726 | 0.593 |
| 10 | 5 | 16 | 11.5 | | | 1.592 | 0.0462 | 0.984 |
| | | 17 | 6.96 | | | 2.109 | 1.262 | 0.667 |
| | | 18 | 4.87 | | | 1.221 | 1.163 | 1.04 |
| 20 | 10 | 19 | 11.5 | | | 3.83 | 1.118 | 5.4 |
| | | 20 | 7 | | | 8.82 | 5.45 | 0.509 |
| | | 21 | 5 | | | 1.306 | 1.173 | 0.1 |
| 30 | 15 | 22 | 11.28 | | | 0.4 | 0.321 | 1.15 |
| | | 23 | 7.1 | | | 1.029 | 0.486 | 0.79 |
| | | 24 | 5.1 | | | 1.365 | 0.272 | 1.07 |
| 5 | 2.5 | 25 | 12.05 | | | 0.51 | 0.1727 | 0.0508 |
| | | 26 | 8.02 | | | NR | NR | NR |
| | | 27 | 4.86 | | | 0.682 | 0.0971 | 0.000265 |
| 10 | 5 | 28 | 12.5 | | | 0.902 | 0.315 | 0.0251 |
| | | 29 | 6.98 | | | 0.421 | 0.2949 | 0.0933 |
| | | 30 | 4.8 | | | 0.41 | 0.0667 | 0.179 |
| 20 | 10 | 31 | 12.67 | | | 0.718 | 0.597 | 0.00839 |
| | | 32 | 7.14 | | | 0.836 | 0.311 | 0.0384 |
| | | 33 | 4.4 | | | 0.41 | 0.359 | 0.102 |
| 30 | 15 | 34 | 12.67 | | | 1.048 | 0.618 | 0.0068 |
| | | 35 | 7.12 | | | NR | NR | NR |
| | | 36 | 4.6 | | | 0.2702 | 0.0869 | 0.00289 |

TABLE 3d

| ALU.BRI.010 | | | | | | | |
|---|---|---|---|---|---|---|---|
| AlCl₃•6H₂O | NaOH | AlCl₃•6H₂O | NaOH | | | | |
| conc | conc | conc | conc | | | | |
| mg/mL | mg/mL | mol/ml | mol/ml | mixing ratio | | molar excess | QaIu |
| 20 | 5 | 8.28397E−05 | 0.000125 | 1 | 2 | 1.005958333 | 2.5 |
| | | | | | | | 5 |
| | | | | | | | 10 |
| | | | | | | | 20 |
| 20 | 6.25 | 8.28397E−05 | 0.000156 | 1 | 2 | 1.257447917 | 2.5 |
| | | | | | | | 5 |
| | | | | | | | 10 |
| | | | | | | | 20 |
| 20 | 7.5 | 8.28397E−05 | 0.000188 | 1 | 2 | 1.5089375 | 2.5 |
| | | | | | | | 5 |
| | | | | | | | 10 |
| | | | | | | | 20 |

| | | | citrate | MV | Mn | |
|---|---|---|---|---|---|---|
| QNaOH | sample number | pH | pH adjusted | μm | μm | loading |
| 5 | 1 | 9.97 | | 1.315 | 0.435 | 0.612 |
| | 2 | | 5.75 | NR | NR | NR |
| | 3 | | 4.75 | 2.503 | 0.921 | 0.065 |
| 10 | 4 | 10.12 | | 2.092 | 0.0923 | 0.001 |
| | 5 | | 6.33 | 1.298 | NR | NR |
| | 6 | | 4.5 | 1.65 | NR | NR |
| 20 | 7 | 10.01 | | 1.942 | NR | NR |
| | 8 | | 4.7 | 3.55 | 1.025 | 0.304 |
| | 9 | | 3.9 | 1.704 | NR | NR |
| 40 | 10 | 10.1 | | 5.03 | 0.307 | 0.000796 |
| | 11 | | 6.5 | 1.205 | NR | NR |
| | 12 | | 4.6 | 2.546 | 0.403 | 0.00893 |
| 5 | 13 | 11.4 | | 0.739 | 0.402 | 0.000677 |
| | 14 | | 6.8 | 1.046 | 0.0428 | 0.216 |
| | 15 | | 4.85 | 1.886 | 1.569 | 0.929 |
| 10 | 16 | 11.4 | | 1.161 | 0.57 | 0.056 |
| | 17 | | 6.85 | 2.052 | 1.216 | 0.755 |
| | 18 | | 4.49 | 2.053 | 0.0775 | 0.34 |
| 20 | 19 | 11.59 | | 1.591 | 0.2722 | 0.131 |
| | 20 | | 6.78 | 1.938 | 1.09 | 0.824 |
| | 21 | | 5.13 | 1.945 | 0.1646 | 0.0963 |
| 40 | 22 | 11.57 | | 0.617 | 0.584 | 0.0339 |
| | 23 | | 6.95 | 2.438 | 0.071 | 0.036 |
| | 24 | | 4.8 | 0.305 | 0.02128 | 0.134 |
| 5 | 25 | 12.68 | | 0.67 | 0.431 | 0.00396 |
| | 26 | | 6.98 | 0.253 | 0.1351 | 0.0297 |
| | 27 | | 5.1 | 0.1393 | 0.0788 | 0.0004 |
| 10 | 28 | 12.5 | | 0.38 | 0.0464 | 0.0005 |
| | 29 | | 6.98 | 0.976 | 0.1295 | 0.152 |
| | 30 | | 5.1 | 0.323 | NR | 0.00008 |
| 20 | 31 | 12.5 | | 0.563 | 0.2273 | 0.000374 |
| | 32 | | 6.99 | 0.368 | 0.0682 | 0.00009 |
| | 33 | | 5.15 | 0.36 | 0.1236 | 0.000297 |
| 40 | 34 | 12.6 | | 0.815 | 0.088 | 0.000524 |
| | 35 | | 7.1 | 0.1558 | 0.084 | 0.000297 |
| | 36 | | 5.1 | 0.364 | 0.146 | 0.000121 |

In test series ALU.BRI.008 it was noted that lowering the stoichiometric excess of NaOH resulted in increasing the loading factor and, despite low particle sizes, dissolution of the particles occurred. A molar ratio of 1 resulted in slightly increased loading factors.

Using a mixing ratio of 2/1 and molar ratios of 1.0 and 1.26, the effects of dissolution were less visible and the product remained more concentrated in the ALU.BRI.009 test series. The loading factor again dropped when applying a molar ratio of 1.5. These samples were opaque and unfilterable, potentially due to the large particle sizes.

In the test series ALU.BRI.010 a mixing ratio of ½ was tested with the different stoichiometric excesses. Particle concentration tended to be low.

### Conclusion

In conclusion, even when adding stoichiometrically equal values, the pH of the completed product was high. pH values above 10 were obtained and therefore dissolution of the product may occur.

### Example 3: Reduction of Final pH and Increasing Particle Concentration

It was considered that it may be possible to increase the concentration of particles by either:
Adding acid to the AlCl₃·6H₂O solution so that the end-pH reaches a value of 7: all AlCl₃ needs to react to produce a full Al(OH)₃ product.
Reduce the stoichiometric excess such that the pH reaches a value of 7.

Experiments were performed to test these hypotheses.

Addition of Acid and Acid Buffer to Decrease pH after Production

Two series of experiments were performed using both citric acid buffer and HCl. Concentrations used were: AlCl₃ 40 mg/mL, NaOH 30 mg/mL, mixing ratio 1/1, molar ratio 1.5, and flow rates 20 mL/min for AlCl₃ and 20 mL/min for NaOH. The results are shown in Table 6 below.

TABLE 6

| | pH before | pH after | Mv | Mn | |
|---|---|---|---|---|---|
| Sample treatment | production | production | μm | μm | Loading |
| No treatment | 3.08 | 13.6 | 0.454 | 0.1086 | 0.00102 |
| 2.5 mL citric acid buffer added to 50 mL of solution | 2.05 | 11.5 | 0.772 | 0.0342 | 0.002 |
| 5 mL citric acid buffer added to 50 mL of solution | 2.43 | 11.94 | 0.00586 | 0.00096 | 0.009 |
| 2 mL of 1N HCl added to 48 mL solution | | 12.0 | 0.729 | 0.0285 | 0.0607 |
| 4 mL of 1N HCl added to 46 mL solution | | 12.3 | 1.507 | 1.311 | 0.276 |
| 6 mL of 1N HCl added to 44 mL solution | | 12.5 | 2.699 | 0.392 | 0.00198 |

Adding citric acid buffer appeared to produce an adverse effect, potentially due to formation of aluminium citrate and therefore a product pH around 7 was not reached. Moreover, the loading factor remained small. The addition of 1N HCl was also not effective, as no visible effect on product pH was found.

### Adjustment of Stoichiometric Ratio to Reach Neutral pH

A titration was performed using a solution of 40 mg/mL AlCl₃·6H₂O and 30 mg/mL NaOH. At a NaOH/AlCl₃ stochiometric ratio of 0.93, a pH value of 7 was reached. This was retested using the same solution concentrations and adjusting the mixing ratio to 1/0.62 (sample ALU.BRI.011 sample 25), rendering flow rates of 20 mL/min for the AlCl₃ flow and 12.4 mL/min for the NaOH flow. As shown in Table 7 below, slightly basic conditions are reached with a pH of 9, however, loading remained low and particle sizes were high. Next, the stochiometric ratio was decreased to 0.86 (ALU.BRI.011 sample 26). At these conditions, a pH value of 6.2 was reached, with a DLS loading factor of 2.11. A cloudy sample was created having about 20 volume % of material around 100 nm in size (based on the volume calculation).

TABLE 7a

| Sample | AlCl₃•6H₂O | NaOH | AlCl₃•6H₂O | NaOH | | | |
|---|---|---|---|---|---|---|---|
| number | conc. | conc. | conc. | conc. | Mixing | | Molar |
| (ALU-BRI-011) | mg/mL | mg/mL | mol/ml | mol/ml | ratio | | ratio |
| 25 | 40 | 30 | 0.000165679 | 0.00075 | 1 | 0.62 | 0.93 |
| 26 | 40 | 30 | 0.000165679 | 0.00075 | 1 | 0.57 | 0.86 |
| 27 | 40 | 30 | 0.000165679 | 0.00075 | 1 | 0.6 | 0.91 |
| 28 | 40 | 30 | 0.000165679 | 0.00075 | 1 | 0.58 | 0.88 |
| 29 | 40 | 17.2 | 0.000165679 | 0.00043 | 1 | 1 | 0.87 |
| 30 | | | | | | | |
| 31 | | | | | | | |
| 32 | | | | | | | |
| 33 | 20 | 30 | 8.28397E−05 | 0.00075 | 1 | 0.287 | 0.87 |
| 34 | 20 | 20 | 8.28397E−05 | 0.0005 | 1 | 0.43 | 0.87 |
| 35 | 20 | 10 | 8.28397E−05 | 0.00025 | 1 | 0.86 | 0.87 |
| 36 | 20 | 8.6 | 8.28397E−05 | 0.000215 | 1 | 1 | 0.87 |

TABLE 7b

| Sample | | Flow | | ph = 5 | | | |
|---|---|---|---|---|---|---|---|
| number | Flow alu. | NaOH | pH | if | MV | Mn | |
| (ALU-BRI-011) | mL/min | mL/min | after | adjusted | μm | μm | loading |
| 25 | 20 | 12.4 | 9.06 | | 1.379 | 1.344 | 0.107 |
| 26 | 20 | 11.4 | 6.2 | | 1.298 | 0.0891 | 2.11 |
| 27 | 20 | 12 | 8.22 | | 1.06 | 0.49 | 0.053 |
| 28 | 20 | 11.6 | 5.61 | | 1.709 | 0.0466 | 1.08 |
| 29 | 20 | 20 | 6.56 | | 1.613 | 0.124 | 2.78 |
| 30 | | | | 4.82 | 2.103 | 0.076 | 1.83 |
| 31 | | | filtr 29 | — | 0.1719 | 0.0915 | 0.378 |
| 32 | | | filtr 30 | | 0.1752 | 0.0591 | 2.23 |
| 33 | 20 | 5.74 | 6.62 | | 1.102 | 0.0605 | 1.44 |
| 34 | 20 | 8.6 | 6.1 | | 1.108 | 0.0744 | 1.46 |
| 35 | 20 | 17.2 | 6.51 | | 1.525 | 0.0353 | 2.38 |
| 36 | 20 | 20 | 5.82 | | 1.139 | 0.0308 | 0.811 |

In ALU.BRI.011 sample 27, the molar ratio of 0.91 resulted in a high pH value of 8.22. Sample 28 with a molar ratio of 0.88 resulted in a pH of 5.61, a good loading value and nearly identical DLS result. Therefore, a stoichiometric ratio of NaOH/AlCl₃, between 0.86 and 0.88 (i.e. ideally around 0.87) was found to be most effective. To test this, and to use a mixing ratio of 1/1 and flow rates of 20 mL/min from both inputs, the concentration of NaOH was adjusted to 17.2 mg/mL (sample 29). This rendered a sample at a pH of 6.56, high loading factor of 2.78 and a bimodal particle size distribution (see FIG. 1). Around 40% of the sample contained particles of around 100 nm diameter.

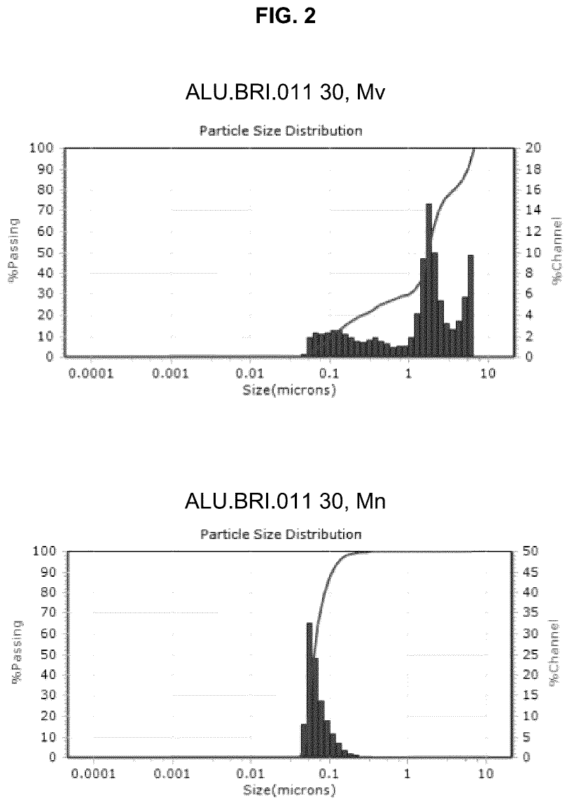

Citric acid buffer was added to this product to prepare a sample at lower pH, sample 30. A pH value of 5.82 was prepared. This initially resulted in the loss of true nanometric particles by approximately 20 volume %, as can be observed in FIG. 2. Both samples 29 and 30 show some opacity. These samples were filtered to check the resultant loading factor (and thus concentration of particles) measured with the DLS and to ascertain how much material (if any) would be lost in filtration.

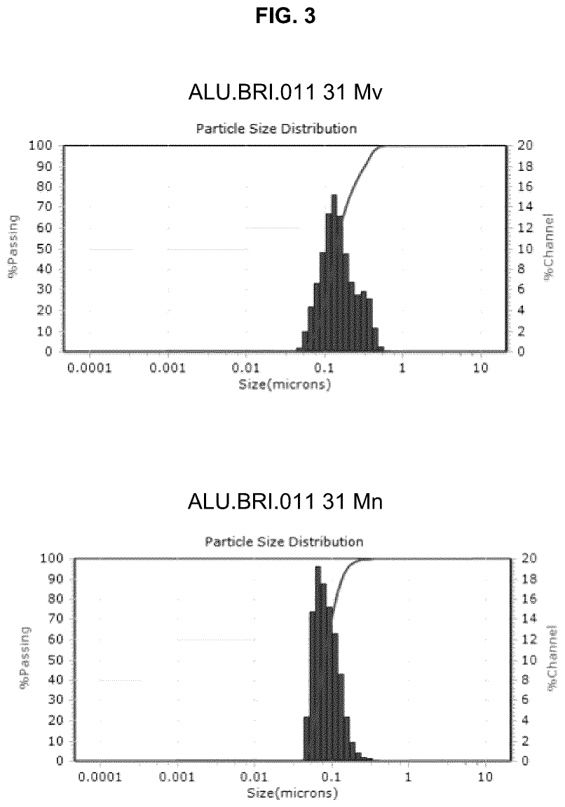

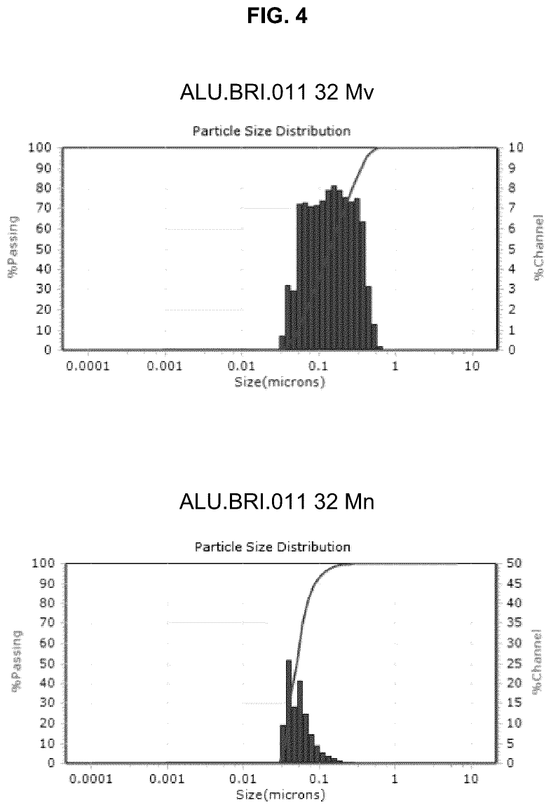

Samples ALU.BRI.011 31 and 32 were created, resulting from filtration with a 0.45 um filter each, originating from sample 29 and 30, respectively. For sample 29, a fully transparent sample was obtained, resulting in a desirable product having both Mv and Mn values close to each other around 110 nm and a loading factor of 0.378. This value is lower than for the initial sample 29, but within an acceptable concentration range. Also, the loading factor of sample 32 is in the same range as the loading factor of sample 30. This indicated that the overall particle concentration is not affected by the filtration. Thus, any agglomerations which form in the samples seem to be broken up with passage through a 0.45 μm filter, resulting in a high loading factor and average Mn and Mv values at 170 and 60 nm respectively (see FIG. 3 and FIG. 4).

The molar ratio of 0.87 was also tested on a solution containing 20 mg/mL AlCl₃·6H₂O and different mixing ratios from about 4/1 (sample ALU.BRI.011 33) to 1/1 (sample ALU.BRI.011 36). All four samples resulted in high loading values and sample properties as measured with DLS were similar to the results that were obtained for a concentration of 40 mg/mL as detailed above.

### Conclusion

It was found that a molar ratio of around 0.865 NaOH/AlCl₃ resulted in particle suspensions having both high loading values and low particle sizes.

Samples produced using a molar ratio of around 0.865 retain the presence of some AlCl₃. Accordingly, tests were performed to investigate if the excess AlCl₃ could be removed without increasing pH. It was also established that direct synthesis of particles in the presence of citric acid buffer in the starting material resulted in unstable particles that degrade rapidly.

### Example 3: Dialysis and Surfactant Experiments

The experiments performed in the previous examples identified the ideal stoichiometric ratio between AlCl₃·6H₂O and NaOH to be around 0.865 (mole NaOH)/(mole AlCl₃). However, using this molar ratio results in unreacted AlCl₃ remaining in the product. Therefore it was investigated if the excess AlCl₃ could be eliminated using a dialysis step.

For dialysis, a cassette was used with a molecular cutoff of 3.5 KD. For all measurements, the product was created by injecting both solutions inside a T-junction mixer and 50 cm long reactor using syringe pumps and stainless steel 50 mL syringes. The product was gathered and 35 mL of sample was used to fill a dialysis cassette. This cassette was prewetted in the designated buffer. After filling the cassette with the product it was kept overnight at room temperature while gently stirring the buffer and cassette with a magnetic bar. After this period the material was evacuated. Depending on the test, the material was then treated with polysorbate 20, filtered (filtering conditions are stated alongside the tests) and pH adjusted.

Different dialysis buffers were tested. Starting concentrations were 40 and 20 mg/ml AlCl₃·6H₂O and therefore 17.2 and 8.6 mg/mL NaOH were used, respectively. The different buffers applied in this test series were:
Salt (about 15 mg/mL concentration)
Succinic acid buffer, 20 mM, adjusted to pH 6.5
Histidine buffer, 20 mM, adjusted to pH 6.5
Phosphate buffer (sodium phosphate and imidazole) of 20 mL, pH adjusted to 6.5
Citric acid buffer, 20 mM, adjusted to pH 6.5.

All samples are doubled in volume with and without dialysis with polysorbate 20 at 0.1 weight % to establish the influence of polysorbate 20 on the stability of the produced particles. Samples were also created applying filtration after a dialysis step, in this case also polysorbate 20 was added. The conditions used are set out in Table 8 below.

TABLE 8

| | |
|---|---|
| Concentration AlCl₃•6H₂O | 40 or 20 mg/mL |
| Concentration NaOH | 17.2 or 8.6 mg/mL |
| Insert and reactor | T-junction - 50 cm reactor |
| Flow rates | 20 mL/min for both sides |
| Mixing ratio | 1:1 |
| Molar ratio | 0.865 (NaOH/AlCl₃) |
| Starting volume | 50 mL + 50 mL |
| Dialysis Cassette | 3500 K MWCO |
| Filters | 0.22, 0.45 μm and 0.8 μm |
| | cellulose acetate |

The different experiments are detailed in Table 9 below. Nearly every sample lost some nanometric characteristics during the dialysis: the DLS loading factor—which is a direct indication for particle concentration—dropped after applying a dialysis treatment with salt, succinic acid buffer, phosphate buffer and histidine buffer.

As an exception, citrate buffer did not degrade the produced nanoparticles. It has no apparent effect on the loading factor (sample ALU-BRI-016 27 had a loading factor above 3 whereas sample ALU-BRI-016 1 had a loading factor of 1.5). Furthermore, when sample ALU-BRI-016 27 was filtered, the measured average sizes dropped, whereas the DLS loading still increased to a value of 5.03 for ALU-BRI-016 28. The obtained pH was at 8.6, while the starting pH was about 5.4 and the applied buffer a pH of 6.5. Therefore, the citrate buffer influenced the behavior of the material and stabilized the particles. The use of citric acid also seemed to aid the decoagulation of the particles upon filtration, something that did not happen by using any other buffer material for the dialysis.

The samples created with citric acid were tested for their behavior using a 0.22 μm filter. For these tests a sample that underwent dialysis with citric acid buffer (ALU-BRI-016 27) and a sample that underwent dialysis and filtration with a 0.45 μm filter (ALU-BRI-016 28) were filtered using a 0.22 μm filter. Again, substantially all material passed through the filter and no pressure build-up to squeeze the material through the filter was felt by the operator. Also, the loading values for these tests, ALU-BRI-016 33 and 34 were adequately high.

To test medium-term stability of the material, samples ALU-BRI-016 27, 28, 33 and 34 were retested after 3 days. Sample sizes and loading values of these tests, also indicated in Table 9, show that the samples remained stable.

The use of polysorbate 20 did not have a clear influence on the decoagulation or stabilization of the material: no influence on stabilization could be observed over this time period.

TABLE 9

| Sample | Filtration | | PS20 | | Dialysis | MV | Mn | |
|---|---|---|---|---|---|---|---|---|
| number | 0.45 μm | Dialysis | 0.1 wt % | pH | buffer | μm | μm | Loading |
| ALU-BRI- | | | | 5.4 | | 2.1 | 0.075 | 1.5 |
| 016 1 | | | | | | | | |
| ALU-BRI- | x | | | 5.4 | | 0.061 | 0.026 | 0.051 |
| 016 2 | | | | | | | | |
| ALU-BRI- | | x | | 4.8 | salt | 0.003 | 0.002 | 0.0069 |
| 016 3 | | | | | | | | |
| ALU-BRI- | x | x | | 4.8 | salt | 0.024 | 0.013 | 0.0009 |
| 016 4 | | | | | | | | |
| ALU-BRI- | | | x | 5.4 | | 1.76 | 0.069 | 1.5 |
| 016 5 | | | | | | | | |
| ALU-BRI- | x | | x | 5.4 | | 0.277 | 0.116 | 0.0688 |
| 016 6 | | | | | | | | |
| ALU-BRI- | | x | x | 4.8 | salt | 0.006 | 0.005 | 0.00353 |
| 016 7 | | | | | | | | |
| ALU-BRI- | x | x | x | 4.8 | salt | 0.021 | 0.021 | 0.0005 |
| 016 8 | | | | | | | | |
| ALU-BRI- | | x | | 7 | phos. | 1 | 0.9 | 0.267 |
| 016 11 | | | | | | | | |
| ALU-BRI- | x | x | | 7 | phos. | NR | NR | NR |
| 016 12 | | | | | | | | |
| ALU-BRI- | | x | x | 7 | phos. | 0.617 | 0.076 | 0.621 |
| 016 15 | | | | | | | | |
| ALU-BRI- | x | x | x | 7 | phos. | 0.016 | 0.004 | 0.0043 |
| 016 16 | | | | | | | | |
| ALU-BRI- | | x | | 6.9 | succinate | 1.2 | 0.249 | 0.0652 |
| 016 19 | | | | | | | | |
| ALU-BRI- | x | x | | 6.9 | succinate | NR | NR | NR |
| 016 20 | | | | | | | | |
| ALU-BRI- | | x | x | 6.9 | succinate | 0.72 | 0.612 | 0.008 |
| 016 23 | | | | | | | | |
| ALU-BRI- | x | x | x | 6.9 | succinate | NR | NR | NR |
| 016 24 | | | | | | | | |
| ALU-BRI- | | x | | 8.6 | citrate | 1.85 | 0.245 | 3.48 |
| 016 27 | | | | | | | | |
| ALU-BRI- | x | x | | 8.6 | citrate | 0.203 | 0.075 | 5.03 |
| 016 28 | | | | | | | | |
| ALU-BRI- | | x | x | 8.6 | citrate | 1.733 | 0.333 | 3.43 |
| 016 31 | | | | | | | | |
| ALU-BRI- | x | x | x | 8.6 | citrate | 0.209 | 0.101 | 5.07 |
| 016 32 | | | | | | | | |
| ALU-BRI- | | x | | | histidine | 2.348 | 0.1343 | 2.64 |
| 018 4 | | | | | | | | |
| ALU-BRI- | x | x | | | histidine | NR | NR | NR |
| 018 5 | | | | | | | | |
| ALU-BRI- | x | x | x | 8.6 | | 0.208 | 0.114 | 4.37 |
| 016 33 | | | | | | | | |
| ALU-BRI- | x | x | | 8.6 | | 0.14 | 0.047 | 2.1 |
| 016 34 | | | | | | | | |
| ALU-BRI- | x | x | x | 8.6 | | 0.199 | 0.085 | 3.69 |
| 016 33 | | | | | | | | |
| retested | | | | | | | | |
| after 3 | | | | | | | | |
| days | | | | | | | | |
| ALU-BRI- | x | x | | 8.6 | | 0.156 | 0.078 | 1.66 |
| 016 34 | | | | | | | | |
| retested | | | | | | | | |
| after 3 | | | | | | | | |
| days | | | | | | | | |
| ALU-BRI- | | x | | 8.6 | | 1.65 | 0.15 | 2.82 |
| 016 27 | | | | | | | | |
| retested | | | | | | | | |
| after 3 | | | | | | | | |
| days | | | | | | | | |
| ALU-BRI- | x | x | | 8.6 | | 0.224 | 0.098 | 3.75 |
| 016 27 | | | | | | | | |
| filtered | | | | | | | | |
| 0.45 after | | | | | | | | |
| 3 days | | | | | | | | |

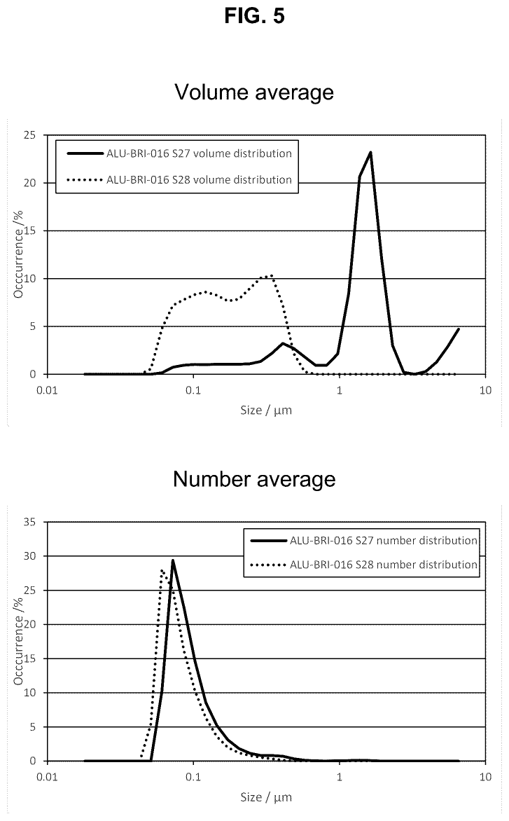

Medium-term stability tests were performed on samples ALU-BRI-016 27, 28, 31, 32, 33 and 34. Measurements were taken after 9 and 15 days. These results, together with the initial measurements are shown in Table 10 below. pH was measured after 9 days and was found for all samples to be slightly above 9. Overall, the samples achieved desirable properties: the non-filtered samples retained the nearly identical Mv sizes above 1 while having Mn values in the 80 to 100 nm range. Filtered samples had a narrow distribution with Mv values of 150 to 200 nm and Mn values again roughly between 80 and 100 nm. Loading factors reduced slightly as a function of time. In FIG. 5 the particle size distribution of ALU-BRI-016 27 and 28 are shown to demonstrate the differences between a filtered and non-filtered sample (ALU-BRI-016 27 is “S27” and is the curve further to the right in both images. ALU-BRI-016 28 is “S28” and is the curve further to the left in both images). The largest portion of particles are under 100 nm. In these images the effect of passage through the 0.45 μm filter (after dialysis) is clearly visible.

TABLE 10

| | Measured after production | | | | Measured after 9 days | | | | Measured after 15 days | | |
|---|---|---|---|---|---|---|---|---|---|---|---|
| | | MV | Mn | | | MV | Mn | | MV | Mn | |
| sample number | pH | μm | μm | loading | pH | μm | μm | loading | μm | μm | loading |
| ALU-BRI- 016 S 27 | 8.6 | 1.85 | 0.245 | 3.48 | 9.12 | 2.4 | 0.082 | 4.03 | 1.56 | 0.0994 | 2.95 |
| ALU-BRI- 016 S 28 | 8.6 | 0.203 | 0.075 | 5.03 | NR | NR | NR | NR | 0.186 | 0.088 | 3.5 |
| ALU-BRI- 016 S 31 | 8.6 | 1.733 | 0.333 | 3.43 | 9.01 | 1.38 | 0.114 | 2.6 | 0.1511 | 0.129 | 2.88 |
| ALU-BRI- 016 S 32 | 8.6 | 0.209 | 0.101 | 5.07 | 9.12 | 0.181 | 0.074 | 3.95 | 0.203 | 0.096 | 3.54 |
| ALU-BRI- 016 S 33 | 8.6 | 0.208 | 0.114 | 4.37 | 9.05 | 0.2 | 0.11 | 3.2 | 0.214 | 0.106 | 3.17 |
| ALU-BRI- 016 S 34 | 8.6 | 0.140 | 0.047 | 2.1 | 9.02 | 0.17 | 0.074 | 1.84 | 0.157 | 0.0843 | 1.37 |

In view of the positive impact of the citric acid buffer dialysis on the nanoparticles, the influence of this buffer was further investigated: retesting of identical conditions was first performed on dialysis cassettes in parallel, citric acid buffer concentrations were changed, and a starting concentration of AlCl₃ was also tested at 20 mg/mL, in test series ALU-BRI-017. The results are shown in Table 11 below.

TABLE 11
AICI3.6 NaOH
Filtration Dialysis

| | | | | | | | | Citric | | | | | | |
|---|---|---|---|---|---|---|---|---|---|---|---|---|---|---|
| | AlCl₃•6H₂O | NaOH | | | | | | acid | Mv | | Mn | | | |
| | conc. | conc. | Filtration | Dial- | | pH after | | buffer | A/B | | A/B | | Loading | |
| Sample number | mg/mL | mg/mL | 0.45 μm | ysis | PS20 | A/B | | conc. | μm | | μm | | A/B | |
| ALU-BRI-017 1 | 40 | 17.2 | | x | | 8.94 | 8.8 | 20 | 1.76 | 2.09 | 0.0775 | 0.118 | 1.75 | 1.75 |
| ALU-BRI-017 2 | 40 | 17.2 | x | x | | 8.97 | 8.94 | 20 | 0.204 | 0.209 | 0.103 | 0.0933 | 2.83 | 2.96 |
| ALU-BRI-017 3 | 40 | 17.2 | | x | x | 8.95 | 8.97 | 20 | 2.084 | 1.336 | 0.156 | 0.191 | 2.99 | 2.4 |
| ALU-BRI-017 4 | 40 | 17.2 | x | x | x | 9.04 | 8.97 | 20 | 0.196 | 0.215 | 0.099 | \|0.117 | 4.83 | 5.1 |
| ALU-BRI-017 5 | 40 | 17.2 | | x | | 7.65 | 7.45 | 40 | 0.703 | 0.26 | 0.043 | 0.046 | 0.048 | 0.035 |
| ALU-BRI-017 6 | 40 | 17.2 | x | x | | 7.48 | 7.65 | 40 | 0.127 | 0.162 | 0.034 | 0.0598 | 0.096 | 0.0992 |
| ALU-BRI-017 11 | 40 | 17.2 | | x | | 7.05 | 7.2 | 10 | 2.256 | 1.928 | 0.137 | 0.117 | 4.13 | 4.96 |
| ALU-BRI-017 12 | 40 | 17.2 | x | x | | unfilterable | | 10 | 0.184 | 0.252 | 0.102 | 0.112 | 0.68 | 2.28 |
| ALU-BRI-017 9 | 20 | \|8.6 | | x | | 8.99 | 9.02 | 20 | 0.764 | 0.911 | 0.067 | 0.075 | 1.27 | 1 |
| ALU-BRI-017 10 | 20 | \|8.6 | x | x | | 9.02 | 9.02 | 20 | 0.195 | 0.174 | 0.082 | 0.078 | 1.53 | 1.46 |

The reproduced samples of this series ALU-BRI-017 1 to 4 are comparable to the results of samples ALU-BRI-016 27, 28, 31, 32: DLS analysis and pH evolution after dialysis show the same tendency and material characteristics. With an increased concentration of the citric acid buffer, to 40 mM, while retaining the pH at 6.5 for dialysis (ALU-BRI-017 5 and 6), the nanometric material disappeared. It appears that the citric acid attacks and dissolves the particles. At too high concentration it dissolves the particle, whereas at around a 20 mM concentration the citric acid stabilizes the material.

Also, when using a citric acid buffer concentration of 10 mM (ALU-BRI-017 11 and 12), this effect is less observed: the sample does not pass as easily through a filter. The filtrate was measured showing good capacities, but the filter blockage indicates that the larger particles cannot be decoagulated during the filtration step as is the case for product that was dialyzed using 20 mM citric acid buffer. The pH of samples dialyzed with both the 40 and 10 mM citric acid concentrations end up at lower pH values, closer to the pH of the buffers used.

The use of a lower concentration of AlCl₃·6H₂O at 20 mg/ml (samples ALU-BRI-017 9 and 10) result in a substantially identical product to the samples with 40 mg/mL starting concentration. A decrease in loading factor is observed for these samples (ALU-BRI-017 9 and 10) as the overall particle concentration is divided by 2. Also varying the concentration of starting product results in a final pH after dialysis slightly above 9.

Medium term storage stability tests were performed for test series ALU-BRI-017. Tests were performed in this case after 10 and 20 days after production. These data are shown in Table 12 below.

TABLE 12

| | Measured after production | | | | Measured after 10 days | | | | Measured after 15 days | | |
|---|---|---|---|---|---|---|---|---|---|---|---|
| | | MV | Mn | | | MV | Mn | | MV | Mn | |
| Sample number | pH | μm | μm | loading | pH | μm | μm | loading | μm | μm | loading |
| ALU-BRI- 017 1a | 8.94 | 1.76 | 0.0775 | 1.75 | 9.11 | 0.832 | 0.056 | 0.917 | 1.43 | 0.056 | 0.763 |
| ALU-BRI- 017 1b | 8.8 | 2.09 | 0.118 | 1.75 | 9.12 | 1.047 | 0.052 | 0.932 | 0.88 | 0.075 | 1.77 |
| ALU-BRI- 017 2a | 8.97 | 0.204 | 0.103 | 2.83 | | 0.154 | 0.070 | 1.65 | 0.141 | 0.053 | 1.29 |
| ALU-BRI- 017 2b | 8.94 | 0.209 | 0.0933 | 2.96 | | 0.170 | 0.069 | 1.65 | 0.1615 | 0.060 | 1.34 |
| ALU-BRI- 017 3a | 8.95 | 2.084 | 0.156 | 12.99 | | 1.034 | 0.056 | 1.82 | 1.355 | 0.066 | 1.84 |
| ALU-BRI- 017 3b | 8.97 | 1.336 | 0.191 | 2.4 | | 0.839 | 0.09 | 1.56 | 1.155 | 0.054 | 1.68 |
| ALU-BRI- 017 4a | 9.04 | 0.196 | 0.099 | 4.83 | | 0.181 | 0.043 | 3.5 | 0.157 | 0.61 | 2.86 |
| ALU-BRI- 017 4b | 8.97 | 0.215 | 0.117 | 5.1 | | 0.188 | 0.089 | 3.61 | 0.183 | 0.086 | 3.11 |
| ALU-BRI- 017 9a | 8.99 | 0.764 | 0.067 | 1.27 | | 0.769 | 0.062 | 0.806 | 0.67 | 0.038 | 1.27 |
| ALU-BRI- 017 9b | 9.02 | 0.911 | 0.075 | 1 | | 0.726 | 0.045 | 0.678 | 0.282 | 0.045 | 0.67 |
| ALU-BRI- 017 10a | 9.02 | 0.195 | 0.082 | 1.53 | | 0.121 | 0.055 | 0.86 | 0.113 | 0.054 | 0.726 |
| ALU-BRI- 017 10b | 9.02 | 0.174 | 0.078 | 1.46 | | 0.125 | 0.055 | 0.814 | 0.114 | 0.049 | 0.679 |

### Conclusion

It was found that dialysis using a citrate buffer could be used to remove the unreacted AlCl₃ which can remain after production of particles using the ideal molar ratio of around 0.865. Using the citric acid buffer during dialysis, after production of the product suspension, resulted in nanometric particles with good stability and did not significantly degrade the nanoparticles nor significantly impact the loading factor.

### Example 4: Achieving Neutral to Slightly Acidic pH Via Addition of Acids after Citric Acid Buffer Dialysis

One of the desirable properties of a nanoalum suspension for use as a vaccine adjuvant is a pH of 6.5 to 7. Previous tests showed that when using a citric acid buffer of 20 mM concentration and a pH of 6.5, all samples over time evolved to a pH of 9 to 9.10. It was unknown whether or not particles produced by the above methods but stored under lower pH conditions would behave well: precipitate rapidly, dissolve, etc. Therefore, tests were performed to adjust pH after dialysis and see whether the particles start to precipitate and if the product after pH adjustment remains stable. Test series ALU-BRI-018 was created and the results are shown in Table 13 below. Standard starting parameters were applied using two different concentrations for AlCl₃ of 40 and 20 mg/mL. Samples of 40 mg/mL were adjusted with 1N HCl to obtain a pH of about 6.5; the samples with 20 mg/mL AlCl₃ were adjusted with histidine buffer to see whether this would give a difference in stability.

Overall, only a few drops of acid resulted in immediate decrease of the pH of the sample, even if the adjustment of the pH was done prior (ALU-BRI-018 2) or after the filtration (ALU-BRI-018 3). At first instance this product appeared to behave well: loading and particle size and size distribution showed normal behaviour. An ageing test was performed 15 days later and these test results are shown in Table 14 below. In this ageing test the loading factor values dropped, and the average particle sizes reduced. This may indicate a dissolution of the product over time. Samples also gradually returned to their initial value of pH 9 after ageing. In the ALU-BRI-019 test series a test was performed under the same conditions, but with 100 mM citric acid buffer to adjust the pH. These measurements, as well as the ageing test after one week are included in Tables 13 and 14 to compare the obtained results. Again, as with the other pH adjustment tests, the pH increased to high values while the product itself degraded, which can again be observed in the lowered particle sizes after ageing, as well as the loading factor decrease.

TABLE 13

| | AlCl₃•6H₂O | NaOH | | | pH after | | with | Mv | | Mn | | | |
|---|---|---|---|---|---|---|---|---|---|---|---|---|---|
| Sample | conc. | conc. | Filtration | Dial- | adjustment | | pH adjusted | A/B | | A/B | | Loading | |
| number | mg/mL | mg/mL | 0.45 μm | ysis | A/B | | mM | μm | | μm | | A/B | |
| ALU-BRI-018 1 | 40 | 17.2 | | x | 6.63 | 6.8 | 1N HCl | 1.05 | 1.586 | 0.075 | 0.075 | 1.18 | 1.12 |
| ALU-BRI-018 2 | 40 | 17.2 | x | x | 6.74 | 6.65 | 1N HCl | 0.188 | 0.163 | 0.092 | 0.086 | 2.56 | 1.65 |
| ALU-BRI-018 3 | 40 | 17.2 | Filtration after | x | 6.63 | 6.8 | 1N HCl | 0.180 | 0.171 | 10.069 | 0.068 | 1.49 | 1.41 |
| | | | adjustment | | | | | | | | | | |
| ALU-BRI-018 6 | 20 | 8.6 | | x | 6.67 | 6.75 | 100 mM His | 0.548 | 1.442 | 0.098 | 0.072 | 0.937 | 0.598 |
| | | | | | | | buffer | | | | | | |
| ALU-BRI-018 7 | 20 | 8.6 | x | x | 6.67 | 6.75 | 100 mM His | 0.167 | 0.166 | 0.061 | 0.063 | 1.11 | 0.412 |
| | | | | | | | buffer | | | | | | |
| ALU-BRI-018 8 | 20 | 8.6 | Filtration after | x | 6.63 | 16.75 | 100 mM His | 0.166 | 0.166 | 0.085 | 0.078 | 0.567 | 0.539 |
| | | | adjustment | | | | buffer | | | | | | |
| ALU-BRI-19 2 | 40 | 17.2 | | x | 6.67 | 16.67 | 100 mM citric | 1.808 | 1.837 | 0.1469 | 0.1174 | 1.69 | 1.83 |
| | | | | | | | acid buffer | | | | | | |
| ALU-BRI-19 2′ | 40 | 17.2 | x | x | | | 100 mM citric | 0.2313 | 0.2776 | 0.0810 | 0.1179 | 2.54 | 2.68 |
| | | | | | | | acid buffer | | | | | | |

TABLE 14

| | pH after | | pH | MV | | Mn | | | |
|---|---|---|---|---|---|---|---|---|---|
| Sample | adjustment | | adjusted | A/B | | A/B | | Loading | |
| number | A/B | | with | μm | | μm | | A/B | |
| ALU-BRI-018 1 | 6.63 | 6.8 | 1N HCl | 1.05 | 1.586 | 0.075 | 0.075 | 1.18 | 1.12 |
| ALU-BRI-018 2 | 6.74 | 6.65 | 1N HCl | 0.188 | 0.163 | 0.092 | 0.086 | 2.56 | 1.65 |
| ALU-BRI-018 3 | 6.63 | 6.8 | 1N HCl | 0.180 | 0.171 | 0.069 | 0.068 | 1.49 | 1.41 |
| ALU-BRI-018 6 | 6.67 | 6.75 | 100 mM His | 0.548 | 1.442 | 0.098 | 0.072 | 0.937 | 0.598 |
| | | | buffer | | | | | | |
| ALU-BRI-018 7 | 6.67 | 6.75 | 100 mM His | 0.167 | 0.166 | 10.061 | 0.063 | 1.11 | 0.412 |
| | | | buffer | | | | | | |
| ALU-BRI-018 8 | 6.63 | 6.75 | 100 mM His | 0.166 | 0.166 | 0.085 | 0.078 | 0.567 | 0.539 |
| | | | buffer | | | | | | |
| AIU-BRI-019 2 | 6.67 | 6.67 | 100 mM | 1.808 | 1.837 | 0.1469 | 0.1174 | 1.69 | 1.83 |
| | | | citric acid | | | | | | |
| | | | buffer | | | | | | |
| ALU-BRI-0192′ | | | 100 mM | 0.2313 | 0.2776 | 0.0810 | 0.1179 | 2.54 | 2.68 |
| | | | citric acid | | | | | | |
| | | | buffer | | | | | | |

| | pH after | | MV | | Mn | | | |
|---|---|---|---|---|---|---|---|---|
| Sample | ageing | | A/B | | A/B | | A/B | |
| number | A/B | | μ | | μ | | Loading | |
| | After two weeks | | | | | | | |
| ALU-BRI-018 1 | 9.02 | 9.04 | 0.366 | 0.182 | 0.036 | 0.0333 | 0.391 | 0.278 |
| ALU-BRI-018 2 | 9.14 | 9.12 | 0.094 | 0.094 | 0.032 | 0.031 | 0.628 | 0.49 |
| ALU-BRI-018 3 | 9.12 | 919 | 0.094 | 0.088 | 0.035 | 0.035 | 0.452 | 0.396 |
| ALU-BRI-018 6 | | | 0.134 | 1.002 | 0.024 | 0.459 | 0.232 | 0.075 |
| ALU-BRI-018 7 | | | 0.059 | 0.087 | 0.028 | 0.038 | 0.19 | 0.129 |
| ALU-BRI-018 8 | | | 0.112 | 0.049 | 0.023 | 0.018 | 10.09 | 0.045 |
| | After one week | | | | | | | |
| AIU-BRI-019 2 | about 9 | | 0.302 | 0.1081 | 0.0465 | 0.0278 | 0.44 | 0.34 |
| ALU-BRI-0192′ | | | 0.0511 | 0.0494 | 0.0294 | 0.0245 | 0.112 | 0.154 |

### Conclusion

It may be concluded that the particle suspension can be adjusted (optionally after dialysis) to the desired pH by adding acids without causing precipitation. Under these conditions, however, the particles can slowly dissolve because they interact with the added molecules, or a new buffer environment is created by adding different acids or more concentrated citric acid buffer.

### Example 5: Reaching the Desired pH Range and Maintaining Low Particle Size by Direct Synthesis in the Presence of Citric Acid Buffer

In a test series to directly synthesize nanoparticles in the presence of citric acid, citric acid buffer was added to the AlCl₃·6H₂O phase prior to mixing. As a mixing ratio of 1 to 1 was applied, initial citric acid buffer concentrations, all at a pH of 6.5, were divided by two for the final product. A total final citric acid buffer concentration range of 10 to 40 mM was envisaged. The goal was to obtain nanometric aluminium hydroxide particles at a pH value of 6.5 to 7. The absence of the dialysis step would be the biggest benefit of this approach.

The use of a citric acid buffer concentration of 10 mM (ALU-BRI-019 3) resulted in a pH of 5.2, the product was fully opaque and settled to the bottom and became unfilterable (ALU-BRI-019 4). The unfiltered material was measured but resulted in a low loading. The use of 20 mM citric acid buffer resulted in a very high pH value. Initially the unfiltered product (ALU-BRI-019 5) had a normal particle size and loading factor. However, upon filtration identical sizes were retrieved (ALU-BRI-019 6), as if the material readily coagulated after formation. The use of both 30 and 35 mM citric acid buffer (ALU-BRI-019 7 to 10) resulted in pH values above 8, the obtained particle sizes before and after filtration in line with previous findings where dialysis was applied. The use of 40 mM citric acid resulted in break-up of the nanoparticles resulting in low particle sizes and low loading factors in the DLS analyses (ALU-BRI-019 11 and 12). After 7 days of ageing, the samples were remeasured. These results are shown in Tables 15 and 16 below.

Samples ALU-BRI-019 7 and 9 (unfiltered product) had reduced Mv values below 1 μm, which indicated possible dissolution of the nanoparticles. This is also shown by the loading factors which dropped to about 50%. The same tendency was observed for the filtered samples (ALU-BRI-019 8 and 10).

TABLE 15

| | | Buffer con- | | | | |
|---|---|---|---|---|---|---|
| | | centration | | | | |
| | Filtra- | after pro- | pH after | | | |
| Sample | tion | duction | pro- | Mv | Mn | |
| number | 0.45 μm | mM | duction | μm | μm | Loading |
| ALU-BRI- | | 10 | 5.2 | 0.827 | 0.01744 | 0.0572 |
| 019 3 | | | | | | |
| ALU-BRI- | x | 10 | 5.2 | NR | NR | NR |
| 019 4 | | | | | | |
| ALU-BRI- | | 20 | 10.2 | 2.986 | 0.2095 | 1.56 |
| 019 5 | | | | | | |
| ALU-BRI- | x | 20 | 10.26 | 2.178 | 0.1412 | 1.36 |
| 019 6 | | | | | | |
| ALU-BRI- | | 30 | 8.7 | 0.775 | 0.0505 | 1.5 |
| 019 7 | | | | | | |
| ALU-BRI- | x | 30 | 8.7 | 0.1782 | 0.0747 | 1.55 |
| 019 8 | | | | | | |
| ALU-BRI- | | 35 | 8.43 | 0.2257 | 0.0482 | 0.659 |
| 019 9 | | | | | | |
| ALU-BRI- | x | 35 | 8.43 | 0.1260 | 0.0558 | 0.504 |
| 019 10 | | | | | | |
| ALU-BRI- | | 40 | 8.6 | 0.1208 | 0.0481 | 0.0733 |
| 019 11 | | | | | | |
| ALU-BRI- | x | 40 | 8.6 | 0.554 | 0.0655 | 0.26 |
| 019 12 | | | | | | |

TABLE 16

| | | Buffer | | | |
|---|---|---|---|---|---|
| | | concentration | | | |
| | | after | | | |
| Sample | Filtration | production | Mv | Mn | |
| number | 0.45 μm | mM | μm | μm | Loading |
| ALU-BRI- | | 10 | NR | NR | NR |
| 019 3 | | | | | |
| ALU-BRI- | X | 10 | NR | NR | NR |
| 019 4 | | | | | |
| ALU-BRI- | | 20 | 4.34 | 0.629 | 0.406 |
| 019 5 | | | | | |
| ALU-BRI- | X | 20 | 3.62 | 0.660 | 0.544 |
| 019 6 | | | | | |
| ALU-BRI- | | 30 | 0.345 | 0.0686 | 0.813 |
| 019 7 | | | | | |
| ALU-BRI- | X | 30 | 0.1905 | 0.0618 | 0.825 |
| 019 8 | | | | | |
| ALU-BRI- | | 35 | 0.1247 | 0.0433 | 0.155 |
| 019 9 | | | | | |
| ALU-BRI- | X | 35 | 0.0894 | 0.0375 | 0.165 |
| 019 10 | | | | | |

| ALU-BRI- | | 40 | Tests not performed. Products |
|---|---|---|---|
| 019 11 | | | were visibly fully degraded |
| ALU-BRI- | X | 40 | |
| 019 12 | | | |

### Conclusion

It was concluded that addition of around 20 mM citric acid buffer to the product suspension during a dialysis step is ideal for stabilising the particles. However, when the concentration of citric acid buffer is too high or the citric acid buffer is already present in the medium before dialysis, it tends to dissolve the product over time. A test series was consequently setup to look to control the eventual pH of the system while retaining particle size. A concentration of below 10 mM does not have sufficient activity to destabilize the larger fraction of particles, whereas concentration above 20 mM destabilizes the particles too much, rendering a fast dissolution of product over time.

### Example 6: Verifying the Ideal Concentration and pH of Citric Acid Buffer

In this test series the objective was to alter the initial pH of the citric acid buffer to see if the pH of the final product after dialysis could be controlled to a value of 6.5 to 7. As the use of a 20 mM citric acid buffer at pH of 6.5 results in a pH of the final product of ~9.10, a range of tests were performed with 20 mM citric acid buffers as dialysis media with a pH ranging between 4 and 6.5. This test series is shown in Table 17 (wherein A/B denotes two repeats, A and B). Besides the anomalous result at pH equal to 5 (ALU-BRI-019 14 and 14′) which resulted in low loading factors, it was observed that the pH range tested after dialysis did not significantly impact loading factors. Also, at buffer pH values below 6, it was challenging to use 0.45 μm filters. Only when using 0.8 um cellulose acetate filters, was it possible to filter the complete product. This resulted in larger average particle sizes for these samples; e.g. ALU-BRI-019 15′. However, this sample had a desirable pH of 6.7.

It appears that adding a too high concentration of citric acid buffer (ALU-BRI-017 5 and 6) degrades the product too far, but adding a too low concentration of citric acid buffer results in an insufficient decoagulation of the product (ALU-BRI-017 11 and 12).

From the previous results described above it appeared that a citric acid buffer at 20 mM concentration and a starting pH at 5.7 should result in a pH of the product suspension after dialysis of around 7. Also, with an initial pH at 5.5 but with higher concentration of citric acid in the buffer, it should be possible to obtain a pH of about 7 and decoagulate the product to obtain nanometric material of the ideal sizes. These samples (ALU-BRI-019 17, 18, 19) are shown in Table 18, also with their evolution over time. Again, using a concentration of citric acid buffer higher than 20 mM resulted in degradation of the product over time, although initially the product with 25 mM citric acid (ALU-BRI-019 18) showed excellent particle size and a desirable pH value after dialysis. After one week this sample had dropped in loading factor, while the pH value increased to a value of 8.9. The product at higher concentration of 30 mM (ALU-BRI-019 19) is completely degraded after one week.

On the other hand, the product with 20 mM citric acid buffer at pH of 5.5 showed excellent properties after production: a pH of 7.34 was measured, together with a filtered material having desired particle sizes (ALU-BRI-019 17).

TABLE 17

| | Filtration | 20 mM | pH | | | | | | | |
|---|---|---|---|---|---|---|---|---|---|---|
| | +pore | citric acid | A/B | | MV | | Mn | | | |
| Sample | size | buffer | After | | A/B | | A/B | | Loading | |
| number | (μm) | pH | dialysis | | μm | | μm | | A/B | |
| ALU-BRI- | | 4 | 4.1 | 4.2 | 1.249 | 1.822 | 0.1225 | 0.1496 | 1.93 | 1.81 |
| 019 13 | | | | | | | | | | |
| ALU-BRI- | X (0.8 | 4 | | | 0.325 | 0.2851 | 0.1171 | 0.0802 | 1.95 | 2.92 |
| 019 13′ | μm) | | | | | | | | | |
| ALU-BRI- | | 5 | 5.5 | 5.45 | 2.144 | 3.38 | 0.0999 | 1.936 | 0.103 | 0.14 |
| 019 14 | | | | | | | | | | |
| ALU-BRI- | X (0.8 | 5 | | | 1.630 | 3.04 | 0.0753 | 0.607 | 0.068 | 0.238 |
| 019 14′ | μm) | | | | | | | | | |
| ALU-BRI- | | 5.5 | 16.67 | 6.7 | 2.236 | 1.816 | 0.1616 | 0.1041 | 2.15 | 2.07 |
| 019 15 | | | | | | | | | | |
| ALU-BRI- | X (0.8 | 5.5 | | | 0.2790 | 0.2938 | 0.0821 | 0.1049 | 2.94 | 3.18 |
| 019 15′ | μm) | | | | | | | | | |
| ALU-BRI- | | 6 | 8.35 | 8.4 | 1.340 | 0.871 | 0.0715 | 0.1198 | 2.28 | 2.62 |
| 019 16 | | | | | | | | | | |
| ALU-BRI- | X (0.45 | 6 | | | 0.2310 | 0.2529 | 0.1146 | 0.0889 | 3.4 | 3.32 |
| 019 16′ | μm) | | | | | | | | | |

TABLE 18

| | | Citric acid | pH | | | | | | |
|---|---|---|---|---|---|---|---|---|---|
| | | buffer | A/B | MV | | Mn | | | |
| Sample | | conc. (mM) | After | A/B | | A/B | | Loading | |
| number | Filtration | and pH | dialysis | μm | | μm | | A/B | |
| ALU-BRI- | | 20 - pH 5.72 | 7.34 | 1.424 | 1.485 | 0.1474 | 0.084 | 1.7 | 1.98 |
| 019 17 | | | | | | | | | |
| ALU-BRI- | X | 20 - pH 5.72 | | 0.1811 | 0.2024 | 0.0628 | 0.0873 | 2.76 | 3.03 |
| 019 17′ | | | | | | | | | |
| ALU-BRI- | | 25 - pH 5.5 | 6.23 | 1.648 | 1.045 | 0.0821 | 0.073 | 1.09 | 0.89 |
| 019 18 | | | | | | | | | |
| ALU-BRI- | X | 25 - pH 5.5 | | 0.1668 | 0.1959 | 0.0634 | 0.0856 | 1.46 | 1.16 |
| 019 18′ | | | | | | | | | |
| ALU-BRI- | | 30 - pH 5.5 | 6.3 | 1.218 | 0.853 | 0.0661 | 0.0459 | 0.517 | 0.34 |
| 019 19 | | | | | | | | | |
| ALU-BRI- | X | 30 - pH 5.5 | | 0.1422 | 0.342 | 0.0499 | 0.0589 | 0.612 | 0.575 |
| 019 19′ | | | | | | | | | |

After storage for 7 days

| | | | | | | | | | |
|---|---|---|---|---|---|---|---|---|---|
| ALU-BRI- | | 20 - pH 5.72 | 8.9 | 1.164 | 1.365 | 0.0694 | 0.0887 | 1.16 | 1.28 |
| 019 17 | | | | | | | | | |
| ALU-BRI- | X | 20 - pH 5.72 | | 0.1669 | 0.171 | 0.0674 | 0.0745 | 1.63 | 1.85 |
| 019 17′ | | | | | | | | | |
| ALU-BRI- | | 25 - pH 5.5 | 8.9 | 0.2583 | 0.387 | 0.0463 | 0.0438 | 0.295 | 0.24 |
| 019 18 | | | | | | | | | |
| ALU-BRI- | X | 25 - pH 5.5 | | 0.0943 | 0.0957 | 0.0445 | 0.0372 | 0.0957 | 0.215 |
| 019 18′ | | | | | | | | | |
| ALU-BRI- | | 30 - pH 5.5 | 9.0 | 0.02022 | 0.35 | | 0.01592 | 0.0259 | 0.076 |
| 019 19 | | | | | | | | | |
| ALU-BRI- | x | 30 - pH 5.5 | | 0.02184 | 10.0308 | 0.01272 | 0.0182 | 0.0127 | 0.0159 |
| 019 19′ | | | | | | | | | |

### Conclusion

It was found that using a high concentration of citric acid buffer as dialysis media significantly degraded the particles, but adding a low concentration of citric acid buffer resulted in an insufficient decoagulation of the particles. Using around 20 mM citric acid buffer at a pH of around 5.5 provided ideal properties after production. Alternatively, the use of 20 mM citric acid buffer dialysis media at pH of around 6.5 results in a particle suspension having ideal particles size after filtration with a 0.45 μm cellulose acetate filter.

In view of all findings presented in the examples above, an ideal protocol has been established as follows:

### A. Preparation of the Starting Liquids

Aqueous solutions of AlCl₃·6H₂O at 40 mg/ml and NaOH at 17.2 mg/ml are prepared. Citric acid buffer at 20 mM is prepared using citric acid and sodium citrate dihydrate, correct concentrations depending on required buffer pH are dissolved in 80% of the total required volume of a stock solution. Once dissolved, the pH is adjusted to the required PH using 1N NaOH or HCl solution. After pH adjustment, water is added to reach the required volume of buffer solution.

### B. Sample Preparation

The sample is prepared by injecting both liquids via stainless steel syringes at a flow rate of 20 mL/min for both liquid phases into a T-junction insert to create the mixture, the liquid passes then straight into a 0.5 mL reactor, after which the product is collected. Sample is then injected into a dialysis cassette with 3500 MWCO. This cassette is first prewetted inside citric acid buffer at the desired concentration and pH. Once the cassette is filled with the product, it is placed back into the citric acid buffer and gently agitated during 15 to 20 hours. The product is then evacuated from the cassette and either placed into a sterile container or filtered using a filter. First a 0.45 um filter could be used, followed by a secondary passage through a 0.22 um filter, if sterility must be ensured.

### Example 7: Long-Term Stability

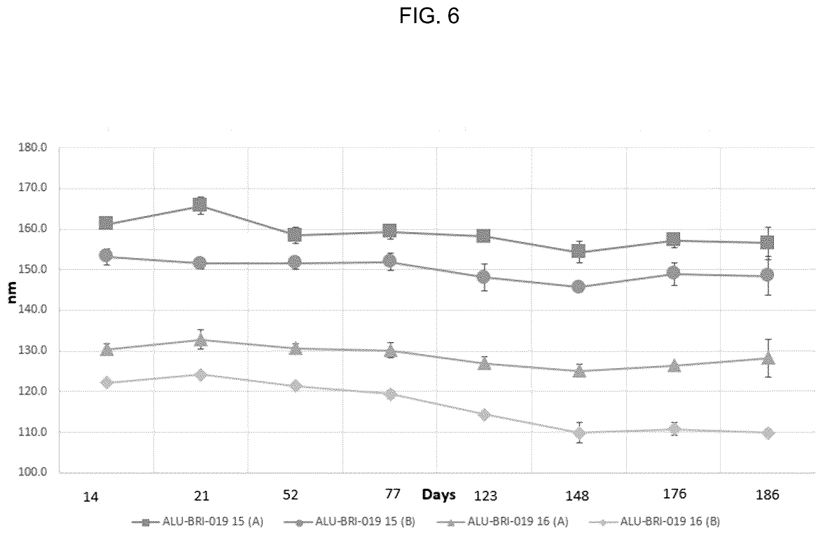

Samples ALU-BRI-019 15 (repeats A and B) and ALU-BRI-019 16 (repeats A and B), each having been filtered with a 0.45 um filter followed by a 0.22 um filter, were tested for their long-term stability. The results of particle size measurements of these samples are shown in FIG. 6.

### Example 8: Impact of Antigen Adsorption

Sample ALU-BRI-019 16 (B) was filtered with a 0.45 um filter followed by a 0.22 um filter. This sample then underwent antigen adsorption and further analysis. The HlaCP5 and ClfACP8 antigens were adsorbed to the aluminium hydroxide particles and the resultant particle diameters were measured. The results are shown in Table 19. It can be seen that particle sizes remained acceptable after antigen adsorption.

TABLE 19

| | Antigen | | | | | Z-average | |
|---|---|---|---|---|---|---|---|
| | dose | Nanoalum | | | | (nm) | |
| | (μg protein- | conc. | Volume | Formul. | Formul. | | 0.22 um |
| Antigen | based) | (mg/ml) | dose | volume | buffer | Unfiltered | filtered |
| HlaCP5 | 10 | 0.5 | 50 μL | 500 μL | 10 mM | 128 ± 2 | 116 ± 2 |
| | | | | | Histidine | | |
| | | | | | pH 6.5 | | |
| | | | | | 8% w/v | | |
| | | | | | Sucrose | | |
| ClfACP8 | 10 | 0.5 | 50 μL | 500 μL | 10 mM | 125 ± 2 | 105 ± 1 |
| | | | | | Histidine | | |
| | | | | | pH 6.5 | | |
| | | | | | 8% w/v | | |
| | | | | | Sucrose | | |

In a second experiment, the HlaCP5 antigen was adsorbed again to the aluminium hydroxide particles in a larger formulation volume. To estimate the amount of unabsorbed antigen after aluminium hydroxide particle separation, 0.02 um filtered or centrifuged samples were taken, and the resultant particle diameters were measured and UPLC analysis was performed. The results are shown in Tables 20 and 21. It can be seen that particle sizes remained acceptable after antigen adsorption.

TABLE 20

| | Antigen | | | | | Z-average (nm) | | | RP-UPLC (%) | |
|---|---|---|---|---|---|---|---|---|---|---|
| | dose | Nanoalum | | | | | | 0.22 and | | 0.22- |
| | (μg protein- | conc. | Volume | Formulation | | | 0.22 um | 0.02 um | 0.22 um | 0.02 um |
| Group | based) | (mg/ml) | dose | volume | Formulation buffer | Unfiltered | filtered | filtered | filtered | filtered |
| 1 | 10 | 0.5 | 50 μL | 5000 μL | 10 mM Histidine pH 6.5 | 128 ± | 119 ± | 6 ± | 103.8 ± | 4.3 |
| | | | | | 8% w/v Sucrose | 1 √ | 1 | 1 | 0.1 | |
| 2 | 10 | / | 50 μL | 5000 μL | 10 mM Histidine pH 6.5 | n.a. | n.a | n.a. | n.a. | 89.9 ± |
| | | | | | 8% w/v Sucrose | | | | | 3.7 |

TABLE 21

| | Z avg. | | Derived count | Attenuator |
|---|---|---|---|---|
| Sample | (nm) | Pdl | rate (kcps) | index |
| Particles-HlaCP5 pre- | 128.3 | 0.21 | 19452.2 | 7 |
| 0.22 um filtration | 128.8 | 0.244 | 19611.3 | |
| (three repeats) | 127.3 | 0.238 | 19546.8 | |
| Particles-HlaCP5 post- | 117.5 | 0.208 | 17775.7 | 7 |
| 0.22 um filtration | 119.2 | 0.239 | 17711.4 | |
| (three repeats) | 118.8 | 0.227 | 17663.4 | |
| Particles-HlaCP5 post- | 6.599 | 0.474 | 173.1 | 11 |
| 0.02 um filtration | 5.814 | 0.431 | 158.6 | |
| (three repeats) | 6.233 | 0.455 | 170.6 | |

TABLE 22

| | Antigen | | | | Z-average (nm) | | |
|---|---|---|---|---|---|---|---|
| | dose | Nanoalum | | | | | 0.22 um |
| | (μg protein- | conc. | Volume | Formul. | | 0.22 um | filtered |
| Antigen | based) | (mg/ml) | dose | volume | Unfiltered | filtered | post 1 day |
| HlaCP5 | 10 | 0.5 | 50 μL | 5000 μL | 149 ± 1 | 138 ± 3 | 137 ± 2 |
| ClFACP8 | 10 | 0.5 | 50 μL | 5000 μL | 150 ± 2 | 139 | 138 ± 2 |
| SpA | 10 | 0.5 | 50 μL | 5000 μL | 168 ± 3 | Unsuccessful | Unsuccessful |

In a third experiment, and using a different batch of aluminium hydroxide particles (sample 16a′), the HlaCP5 and ClfACP8 antigens was adsorbed again to the aluminium hydroxide particles in a larger formulation volume, along with SpA antigen. The resultant particle diameters were measured. The results are shown in Table 22. It can be seen that particle sizes remained low after antigen adsorption. The formulation buffer in all cases was 10 mM Histidine pH 6.5, 8% w/v sucrose. It can be seen that particle sizes remained acceptable after antigen adsorption.

### Example 9—Immunogenicity of Aluminium Hydroxide Particle-Containing Formulations

An in vivo study was performed to determine the relative immunogenicity of formulations containing aluminium hydroxide particles of differing sizes. Vaccine formulations comprising nano-size aluminium hydroxide particles (nanoalum) and Hla-CP5 were prepared as described in Example 8 above with autoclave sterilisation. Formulations containing Hla-CP5 antigen and traditional aluminium hydroxide particles with a particle size of ~3000 nm were also prepared with autoclave sterilisation. The formulations were prepared with either 0.1 μg or 1 μg HlaCP5 antigen per dose and with 0.5 mg/ml aluminium hydroxide particles. The formulations were prepared immediately prior to mouse immunisation.

Seven-week old CD-1 mice (16 per group) were each immunised intramuscularly with 50 μl total (25 μl per leg) of the vaccine formulations. Mice were immunised with a first dose of the formulations on day 1, followed by a second dose on day 29. Blood was collected on days 0, 28 (4 weeks post first immunisation) and 43 (two weeks post second immunisation). On day 43, mice were euthanised and spleens were collected.

### Antigen-Specific IgG

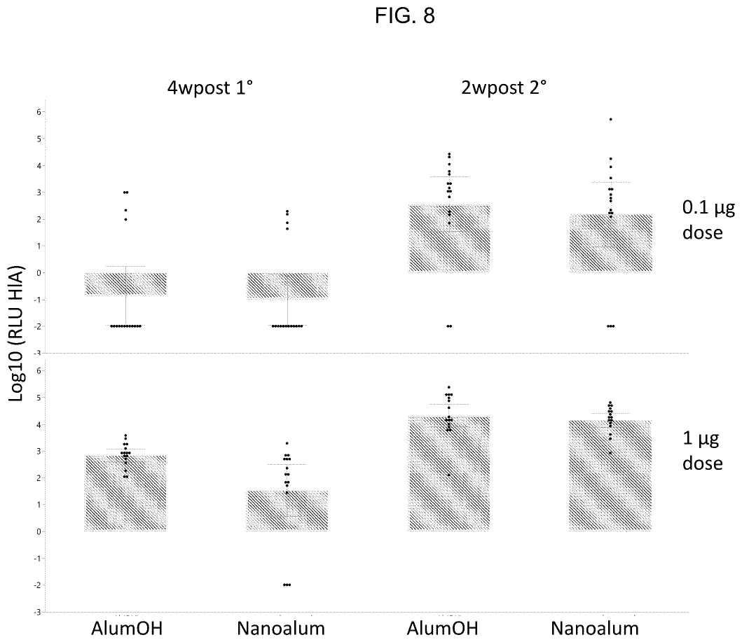

HlaCP5 antigen-specific IgG present in immunised mouse serum was measured using a LUMINEX assay. Briefly, sera were serially 3-fold diluted then each dilution was transferred to a 96-well plate. Beads coated with Hla or CP5 were added to the sample wells (10 μl/well) then incubated in the dark for 1 hour at room temperature. Beads were then washed three times with 100 μl PBS/well before addition of 25 μl/well 1:100 diluted R-Phycoerythrin AffiniPure F(ab′)2 Fragment Goat Anti-Mouse IgG, F(ab′)₂ fragment specific (Jackson 115-116-072) for 15 minutes in the dark at room temperature. Beads were washed three times with PBS then plates were read using a LUMINEX plate reader. In FIGS. 8-11 “AlumOH” refers to formulations comprising traditional aluminium hydroxide particles, “Nanoalum” refers to formulations comprising aluminium hydroxide particles produced as in Example 8, RLU indicates the Relative Light Units, 4wpost 1° indicates testing of sera taken 4 weeks after the first immunisation, and 2wpost 2° indicates testing of sera taken 2 weeks after the second immunisation.

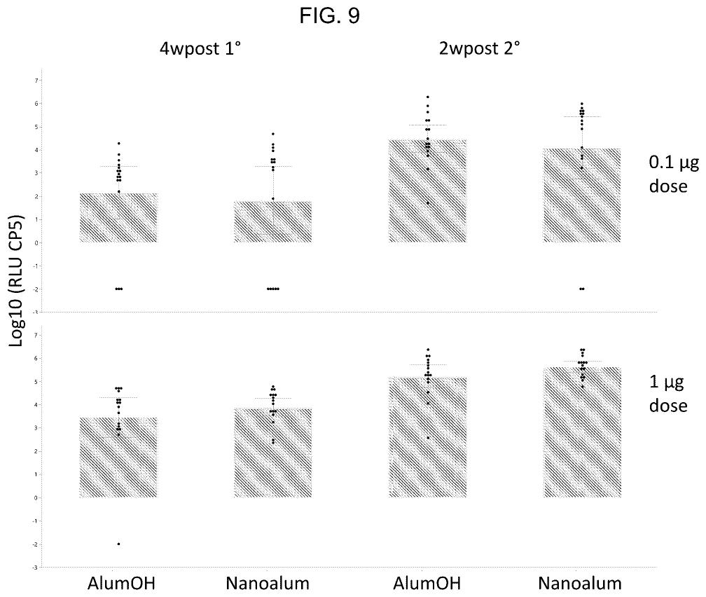

FIG. 8 shows the total Hla antigen-specific IgG detected in mice after the first or second immunisation with either 0.1 μg or 1 μg HlaCP5 antigen formulated with aluminium hydroxide particles. Anti-Hla IgG titres were equivalent between nanoalum and traditional aluminium hydroxide particles. A statistically significant but small difference in antigen-specific IgG titres in mice immunised with 1 μg HlaCP5 was observed between nanoalum and traditional aluminium hydroxide particles after the second immunisation. FIG. 9 shows the total CP5 antigen-specific IgG detected in mice after the first or second immunisation with either 0.1 μg or 1 μg HlaCP5 antigen formulated with aluminium hydroxide particles. Anti-CP5 IgG titres were equivalent between nanoalum and traditional aluminium hydroxide particles.

### Antigen-Specific T Cells

Spleens harvested from euthanised mice were placed in 1.5 ml wash medium (RPMI 1640 (GIBCO Cat. 21875-034)+10% foetal bovine serum+1% penicillin/streptomycin), then crushed and filtered over a 70 um cell strainer. Cells were washed a further two times before addition of 1×RBC lysis buffer (BIOLEGEND) on ice for 2 minutes. Splenocytes were washed then plated in 96-well plates at 2×10⁶ cells/well with Hla peptide pool and 8 μg/ml anti-CD28. Splenocytes cultured with 1 μg/ml anti-CD3 and 8 μg/ml CD28 were used as a positive control. Splenocytes cultured with 8 μg/ml anti-CD28 and medium were used as a negative control. Plates were incubated at 37 °C overnight then 5 μg/ml Brefeldin A was added for 4 hours. Cells were washed twice in PBS then stained with Live/Dead Near-IR for 20 minutes followed by surface staining with anti-CD44-APC-R700 (BD Pharmingen Cat. 565480) and CD62L-BB515 (BD Pharmingen Cat. 565261). Cells were washed and permeabilised in CytoFix/CytoPerm (Becton Dickinson Cat. 554722) at 4 °C and 1× PermWash (Becton Dickinson 554723). Fc block (BD Pharmingen Cat. 553141) was added to cells for 20 minutes in the dark at room temperature then cells were stained with CD45-APC (BD Pharmingen Cat. 559864), CD3-BB700 (BD Pharmingen Cat. 566494), CD4-BUV395 (BD Pharmingen Cat. 740208), CD8-BUV805 (BD Pharmingen Cat. 612898), IFN-γ-BV480 (BD Pharmingen Cat. 566097), TNF-α-BV711 (BD Pharmingen Cat. 563944), IL-2-BV421 (BD Pharmingen Cat. 562969), IL-13-PE (eBioscience Cat. 12-7133-82), IL-17-BV650 (BD Pharmingen Cat. 564170), IL-1β-PE-Cy7 (eBioscience Cat. 25-7114-82) and IL-4-PE (BioLegend Cat. 504104) for 20 minutes at room temperature. Cells were washed once in 1× PermWash, once in PBS, then data were acquired on a LSRII flow cytometer. Gating was performed on CD45+CD3+CD44+CD4+ or CD45+CD3+CD44+CD8+ to determine the percentage of antigen (Ag)-specific cytokine-producing T cells.

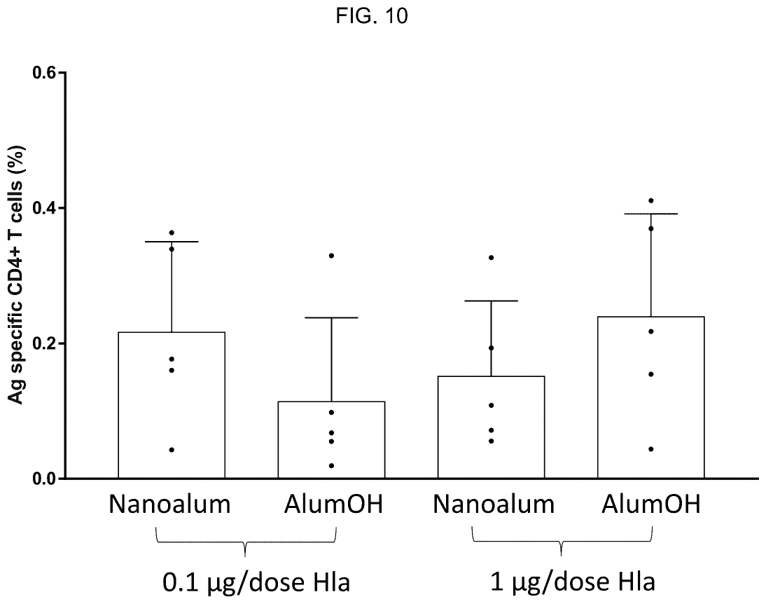

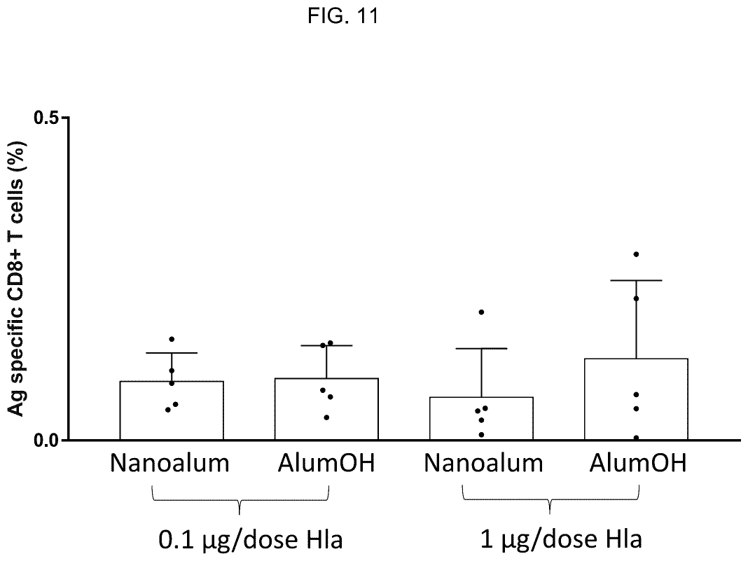

In FIGS. 10-11 “AlumOH” refers to formulations comprising traditional aluminium hydroxide particles, whilst “nanoalum” refers to formulations comprising nano-sized aluminium hydroxide particles produced as in Example 8. FIG. 10 shows the percentage of antigen-specific CD4+ T cells induced following two immunisations with 0.1 μg or 1 μg HlaCP5 formulated with nanoalum or traditional aluminium hydroxide particles. The percentage of induced antigen-specific CD4+ T cells was equivalent between nanoalum and traditional aluminium hydroxide particles.

FIG. 11 shows the percentage of antigen-specific CD8+ T cells induced following two immunisations with 0.1 μg or 1 μg HlaCP5 formulated with nanoalum or traditional aluminium hydroxide particles. The percentage of induced antigen-specific CD8+ T cells was equivalent between nanoalum and traditional aluminium hydroxide particles.

### Example 10—Characterization of Size and Morphology of Aluminium Hydroxide Particles

Nanoparticle Tracking Analysis (NTA) and cryogenic Electron Microscopy (cryo-EM) were used to characterize the size and morphology of nano-sized aluminium hydroxide particles. A sample comprising aluminium hydroxide particles was prepared according to the ‘ideal protocol’ described in Example 6.

### NTA

The NTA instrument Zeta VIEW (Particle Metrix) was used to measure size-distribution (per particle) and particle concentration. The particle diffusion was tracked using a 488 nm laser >30 mW across 11 focal positions with a known cell chamber volume. The scattering signal from the particles was then recorded using a 640×480 pixels CMOS camera. The sample was diluted 10 000 times in ddH2O to a final volume of 2 mL prior analysis to achieve an optimal number of particles per focal position (300-400 particles/frame, higher limit of the equipment). A specific standard operating procedure (SOP) was designed to optimise the detection of particles for this sample with the following parameters: shutter 150, intensity 80, temperature 22 °C. After the video capture, data were captured and analysed using the ZetaVIEW software (Particle Metrix) with default settings.

Prior to the experiment, a daily performance check using auto alignment and autofocus of the NTA Zeta VIEW system was done with the calibration polystyrene beads 100 nm (Applied Microspheres Nanostandards 0,100 nm, provided by Particle Metrix).

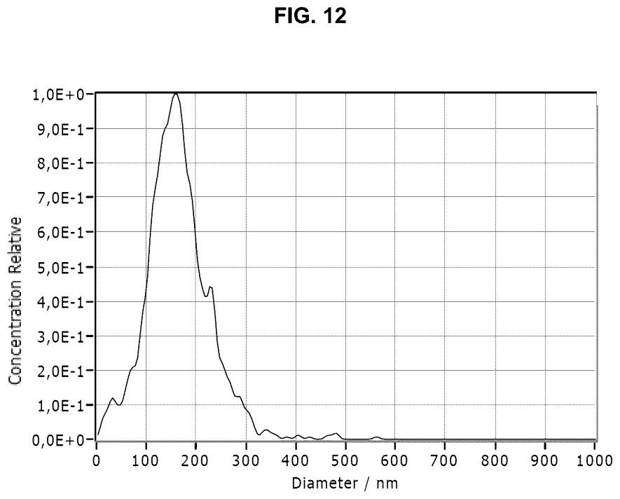

NTA results indicated that the sample contained an average aluminium hydroxide particle concentration of 1.75×10¹² particles/mL and a median particle diameter around 150 nm (Table 23, FIG. 12).

TABLE 23
Median (X50) size-distribution of particles and total particle
concentration across 4 technical repeats as determined by NTA

| | Median | Std | Total concentration |
|---|---|---|---|
| | (X50) (nm) | Dev (nm) | (particles/mL) |
| run1 | 152.3 | 62.5 | 1.8E+12 |
| run2 | 156.9 | 63.2 | 1.7E+12 |
| run3 | 152.9 | 65.8 | 1.7E+12 |
| run4 | 149.9 | 61.2 | 1.8E+12 |

### Cryo-EM

4 μL of undiluted sample was added on a glow discharged carbon-coated grid Quantifoil 300 Mesh 2/2 Copper (Quantifoil) and incubated for 5 seconds prior to blotting. Excess liquid sample was blotted off the grid using blotting paper (Whatman No. 1) on a Vitrobot Mark IV (ThermoFisher Scientific). A blotforce of 3 and blot time of 4 seconds in a 100% humidity chamber were used. The grid was then directly mounted into a side-entry cryo-holder Gatan 626 (Gatan Inc.) and inserted into a Talos F200C (ThermoFisher Scientific) transmission electron microscope in cryogenic condition for observation. Pictures were acquired using a 4/4k CetaD CMOS camera with a cumulated electron dose of 30 e−/A². Data acquisition was performed using Velox software (ThermoFisher Scientific) at different magnifications ranging from ×17500 to ×28000 (0.61 nm/pixel and 0.36 nm/pixel respectively) and at different defocus values ranging from −5 to −20 um according to the magnification used. Particles counting and size estimation were manually realised using Fiji ImageJ suite software (Fiji ImageJ v1.51).

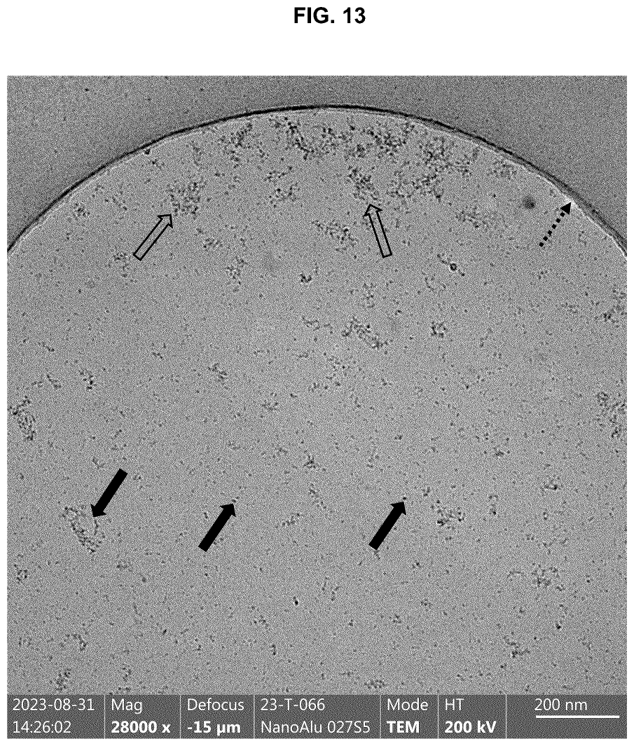

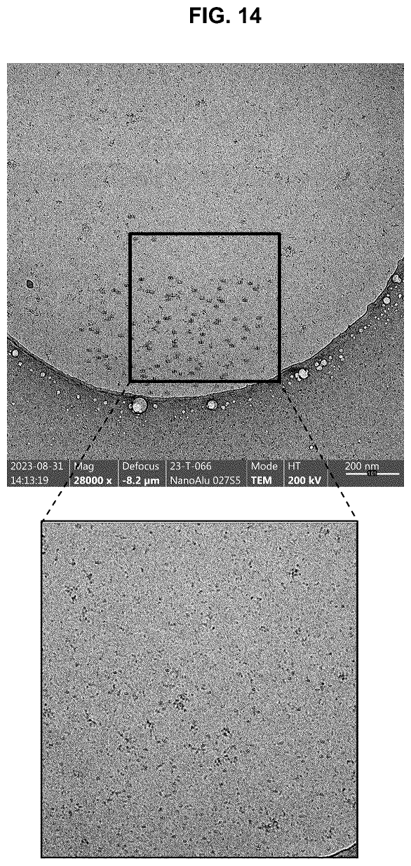

Observations of large clusters of nanoparticles with an estimated size range between 200-400 nm could be observed in Cryo-EM. Additionally, smaller nanoparticles could also be observed (FIG. 13, FIG. 14). A size estimation of the smallest detected nanoparticles was obtained manually in ImageJ giving a value of 4.70±0.91 nm (N=100) which is ×26 smaller than the average size obtained by DLS and NTA. However, it has to be taken into account that the particle distribution observed by cryo-EM could be influenced by the grid preparation and by the physical and chemical properties of the sample used. With a lower limit of detection, it is suggested that unlike DLS or NTA, cryo-EM can detect particles having diameters <40 nm.

### REFERENCES

Fifis et al. Size-dependent immunogenicity: therapeutic and protective properties of nano-vaccines against tumors, J. Immunol. 173 (2004) 3148-3154.
Li et al. Relationship between the size of nanoparticles and their adjuvant activity: Data from a study with an improved experimental design European Journal of Pharmaceutics and Biopharmaceutics 78 (2011) 107-116.
Li et al. Aluminum hydroxide nanoparticles show a stronger vaccine adjuvant activity than traditional aluminum hydroxide microparticles Journal of Controlled Release 173 (2014) 148-157.
WO2019215022

1. A method of making a composition; the composition comprising aluminium hydroxide particles; the method comprising reacting an aluminium ion-containing compound with a hydroxide ion-containing compound in a micro-fluidic system or a milli-fluidic system, thereby obtaining the composition.

2. The method of claim 1, wherein the aluminium ion-containing compound is AlCl₃ and the hydroxide ion-containing compound is NaOH.

3. The method of claim 1, wherein the aluminium ion-containing compound and the hydroxide ion-containing compound are each in an aqueous solution.

4. The method of claim 1, wherein the reacting comprises a molar ratio of the hydroxide ion-containing compound to the aluminium ion-containing compound from 0.700:1 to 1.000:1.

5. The method of claim 1, wherein the reacting comprises a volume mixing ratio per unit time of the aluminium ion-containing compound to the hydroxide ion-containing compound from 2:1 per unit time to 1:2 per unit time.

6. The method of claim 1, wherein the composition comprises a concentration of the aluminum ion-containing compound and, the method further comprises, after the reacting, dialyzing the composition to reduce the concentration of the aluminium ion-containing compound in the composition, thereby obtaining a dialyzed composition.

7. The method of claim 6, wherein the dialyzing comprises a dialysis membrane, the dialysis membrane having a molecular cut-off of 2.5 to 4.5 kDa.

8. The method of claim 6, wherein the dialyzing comprises a dialysis medium, and the dialysis medium comprises a citric acid buffer.

9. The method of claim 8, wherein the dialysis medium comprises 10 mM to 30 mM of the citric acid buffer.

10. The method of claim 8, wherein the citric acid buffer has a pH of from 5.3 to 6.7.

11. The method of claim 1, wherein the aluminium hydroxide particles have: (1) a volume distribution mean diameter of 0.500 μm or lower, (2) a number distribution mean diameter of 0.500 μm or lower, or (3) both (1) and (2).

12. The method of claim 1 further comprising filtering the composition after the reacting.

13. The method of claim 12 wherein the composition is filtered with a 0.45 μm filter.

14. The method of claim 1, wherein the composition has a pH of from 6.5 to 7.0.

15. The method of claim 6 further comprising adding a histidine buffer to the dialyzed composition.

16. The method of claim 1 comprising the microfluidic system; the microfluidic system comprising a first inlet, a second inlet, an outlet, and mixing chamber; the first inlet and second inlet being in fluid communication with the mixing chamber; the mixing chamber being in fluid communication with the outlet; the aluminum ion-containing compound flowing through the first inlet into the mixing chamber; and the hydroxide ion-containing compound flowing through the second inlet into the mixing chamber.

17. The method of claim 16, wherein the mixing chamber has a diameter of 500 μm.
